(19) United States
(12) Patent Application Publication
(10) Pub. No.: US 2023/0330385 A1
(43) Pub. Date: Oct. 19, 2023

(54) AUTOMATED BEHAVIOR MONITORING AND MODIFICATION SYSTEM

(71) Applicant: MindfulGarden Digital Health, Inc., Vancouver (CA)

(72) Inventors: Catherine Winckler, Vancouver (CA); Mark Ross, Delta (CA); Nicolas Shuster, West Vancouver (CA)

(21) Appl. No.: 18/133,619

(22) Filed: Apr. 12, 2023

Related U.S. Application Data
(60) Provisional application No. 63/330,448, filed on Apr. 13, 2022.

(51) Int. Cl. A61M 21/02

(52) U.S. Cl. CPC A61M 21/02; A61M 2021/0027

(57) ABSTRACT

The invention provides automated interactive behavior monitoring and modification systems designed to arrest and de-escalate agitated behaviors in a patient.

### CROSS-REFERENCE TO RELATED APPLICATIONS

This application claims priority to, and the benefit of, U.S. Provisional Application No. 63/330,448, filed Apr. 13, 2022, the content of which is incorporated by reference herein in its entirety.

### TECHNICAL FIELD

The invention relates to a system for monitoring patient behavior and subsequently providing automated audible and/or visual content to the patient for modulating any disruptive behavior, particularly those related to nervous system diseases, neurocognitive disorders, and/or mental disorders.

### BACKGROUND

Disruptive behaviors, particularly those associated with mental disturbances, can become a significant strain on healthcare resources, particularly in terms of staff, as well as physical and financial resources. Delirium, for example, affects as many as 80% of patients in critical care. Delirium is an acute neuropsychiatric condition of fluctuating confusion and agitation. The clinical presentation of delirium is variable, but can be classified broadly into three subtypes on the basis of psychomotor behavior, which include: hypoactive; hyperactive; and mixed. Patients with hyperactive delirium demonstrate features of restlessness, agitation and hyper vigilance and often experience hallucinations and delusions. For those patients suffering from hyperactive delirium, associated behavior can become aggressive and/or combative, putting both themselves and healthcare workers at risk of harm. By contrast, patients with hypoactive delirium present with lethargy and sedation, respond slowly to questioning, and show little spontaneous movement. Patients with mixed delirium demonstrate both hyperactive and hypoactive features.

Delirium is further associated with an increased risk of morbidity and mortality, increased healthcare costs, and adverse events that lead to loss of independence and poor overall outcomes. For example, hospitalized delirium is prevalent in the ICU at a rate of 60-80%. Patients hospitalized with delirium have twice the length of stay and readmission, and three times the rate of mortality, as compared to those patients without. The healthcare costs associated with delirium are substantial, rivaling costs associated with cardiovascular disease and diabetes, for example.

Current methods for treating delirium require significant and ongoing commitments of skilled staffing, volunteer resources, and financial support. In particular, for non-pharmacological approaches, patients generally require one-on-one attendance from a caregiver who provides reorientation and/or behavioral intervention strategies. Treatments may further require pharmacological interventions, as well as the use of physical restraints, which have been linked to adverse outcomes and significant side effects. As a result, addressing the over prescribing of psychotropic drugs and restraints is a global healthcare priority. Clinicians are still searching for effective strategies to ensure the best possible outcomes for patients, and there remains a need for effective interventions for patients with disruptive behavior, particularly those associated with delirium and other nervous system diseases, neurocognitive disorders, and mental disorders.

### SUMMARY

The present invention recognizes the drawbacks of current clinical protocols in managing and modifying disruptive behaviors associated with a nervous system disease, neurocognitive disorder, and/or mental disorder. More specifically, the present invention recognizes the limitations of both non-pharmacological and pharmacological management programs, particularly in terms of the significant and on-going requirement of skilled staffing, volunteer resources, and financial support necessary for each patient.

To address the drawbacks of current treatment methods, the invention provides an automated interactive behavior monitoring and modification system designed to arrest and de-escalate agitated behaviors in the patient. Aspects of the invention may be accomplished using a platform configured to receive and analyze patient input, and, based on such analysis, present audible and/or visual content to the patient to reduce anxiety and/or agitation in the patient. In doing so, normalization of agitation and delirium scores can be achieved without the reliance on pharmacological interventions or the use of physical restraints.

The platform utilizes various sensors for capturing a patient’s activity, which may include patient motion, vocalization, as well as physiological readings. Therefore, the various sensors are able to capture a wide spectrum of the patient’s behavior at a given point in time, thereby providing data points, in real time, of a patient’s distress level. In turn, based on captured patient activity data, the platform is able to output corresponding levels of audible and/or visual content as a means of distracting and/or engaging the patient so as to ultimately deescalate a patient’s distress level. As the anxious and aggressive behaviors calm, so too does the output, reducing the agitation levels of the patient and making the patient more receptive to care.

In one aspect, the invention provides a system for providing automated behavior monitoring and modification in a patient. The system includes an audio/visual device, one or more sensors, and a computing system. The audio/visual device is configured to present audible and/or visual content to a patient exhibiting one or more disruptive behaviors associated with a mental state. The one or more sensors are configured to continuously capture patient activity data during presentation of the audible and/or visual content. The patient activity data may include at least one of patient motion, patient vocalization, and patient physiological readings. The computing system is operably associated with the audio/visual device and configured to control output of the audible and/or visual content therefrom based, at least in part, on the patient activity data. The computing system is configured to receive and analyze, in real time, the patient activity data from the one or more sensors and, based on the analysis, determine a level of increase or decrease in patient activity over a period of time. In turn, the computing system is configured to dynamically adjust a level of output of the audible and/or visual content from the audio/visual device to correspond to the determined level of increase or decrease in patient activity.

In some embodiments, an increase in patient activity may include, for example, at least one of increased patient motion, increased vocalization, and increased levels of physiological readings. Accordingly, the computing system is configured to increase the level of output of the audible and/or visual content to correspond to an increase in patient activity. The increased level of output of the audible and/or visual content may include at least one of: an increase in an amount of visual content presented to the patient; an increase in a type of visual content presented to the patient; and increase in movement of visual content presented to the patient; an increase in a decibel level of audible content presented to the patient; an increase in frequency and/or tone of audible content presented to the patient; and an increase in tempo of audible content presented to the patient.

In some embodiments, a decrease in patient activity may include, for example, at least one of decreased patient motion, decreased patient vocalization, and decreased levels of patient physiological readings. Accordingly, the computing system is configured to decrease the level of output of the audible and/or visual content to correspond to a decrease in patient activity. The decreased level of output of the audible and/or visual content may include at least one of: a decrease in an amount of visual content presented to the patient; a decrease in a type of visual content presented to the patient; a decrease in movement of visual content presented to the patient; a decrease in a decibel level of audible content presented to the patient; a decrease in frequency and/or tone of audible content presented to the patient; and a decrease in tempo of audible content presented to the patient.

In some embodiments, the computing system is configured to dynamically adjust levels of output of the audible and/or visual content based on adjustable predefined ratios applied to patient activity data.

The patient activity continuously captured by the one or more sensors may include patient motion, wherein the patient motion includes facial expressions, physical movement, and/or physical gestures. The patient activity may be physiological readings comprising the patient’s body temperature, heart rate, heart rate variability, blood pressure, respiratory rate and respiratory depth, skin conductance, and oxygen saturation.

The disruptive behaviors associated with a mental state may be, for example, varying levels of agitation, distress, and/or confusion associated with the mental state. In particular, the disruptive behaviors may be associated with delirium. In some embodiments, each of the varying levels of agitation, distress, and/or confusion may be associated with a measured Richmond Agitation Sedation Score and/or a similar clinically-accepted Delirium Score. The Richmond Agitation Sedation Score or the Delirium score may be entered into the computing system by a clinician as input. The system then manages behavioral change by dynamically adjusting the level of output of the audible and/or visual content based on the measured score as input.

The one or more sensors may include one or more cameras, one or more motion sensors, one or more microphones, and/or one or more biometric sensors.

The audible and/or visual content presented to the patient includes sounds and/or images. The images may include two-dimensional (2D) video layered with three-dimensional (3D) animations. The images may include nature-based imagery. Further, the content in the images may be synchronized to the time of day in which the images are presented to the patient. Additionally, the sounds presented to the patient may be noise-cancelling and/or noise-masking.

In another aspect, a computing system for providing automated behavior monitoring and modification in a patient is provided. The computing system includes a hardware processor coupled to non-transitory, computer-readable memory containing instructions executable by the processor to cause the computing system to perform various operations for receiving and analyzing patient activity data and providing audible and/or visual content to a patient exhibiting one or more disruptive behaviors associated with a mental state.

The system includes a computing system for providing automated behavior monitoring and modification in a patient, wherein the computing system includes a hardware processor coupled to non-transitory, computer-readable memory containing instructions executable by the processor to cause the computing system to perform various operations for receiving and analyzing patient activity data to produce a level of output of audible and/or visual content to a patient exhibiting one or more disruptive behaviors associated with a mental state.

In particular, the computing system is configured to receive and analyze, in real time, patient activity data captured by one or more sensors during presentation of audible and/or visual content to a patient exhibiting one or more disruptive behaviors associated with a mental state. The system then determines a level of increase or decrease in patient activity over a period of time based on the analysis, and dynamically adjusts the level of output of the audible and/or visual content to the patient to correspond to the determined level of increase or decrease in patient activity.

The patient activity data may include at least one of patient motion, vocalization, and physiological readings.

An increase in patient activity may be, for example, at least one of increased patient motion, increased vocalization, and increased levels of physiological readings. The computing system is configured to increase the level of output of the audible and/or visual content to correspond to an increase in patient activity. The increased level of output of the audible and/or visual content may include at least one of: an increase in an amount of visual content presented to the patient; an increase in a type of visual content presented to the patient; and increase in movement of visual content presented to the patient; an increase in a decibel level of audible content presented to the patient; an increase in audible frequency and/or tone presented to the patient; and an increase in tempo of audible content presented to the patient.

Likewise, the computing system is configured to decrease the level of output of the audible and/or visual content to correspond to a decrease in patient activity. The decrease in patient activity may include at least one of decreased patient motion, decreased patient vocalization, and decreased levels of patient physiological readings. Further, the computing system is configured to decrease a level of output of audible and/or visual content to correspond to a decrease in patient activity. This decreased level of output may include at least one of: a decrease in an amount of visual content presented to the patient; a decrease in a type of visual content presented to the patient; a decrease in movement of visual content presented to the patient; a decrease in a decibel level of audible content presented to the patient; a decrease in audible frequency and/or tone presented to the patient; and a decrease in tempo of audible content presented to the patient.

Further, the computing system is configured to dynamically adjust levels of output of the audible and/or visual content based on adjustable predefined ratios applied to patient activity data.

The patient activity continuously captured by the one or more sensors may include patient motion, wherein the patient motion includes facial expressions, physical movement, and/or physical gestures. The patient activity may include physiological readings comprising the patient’s body temperature, heart rate, heart rate variability, blood pressure, respiratory rate and respirator depth, skin conductance, and oxygen saturation.

The patient activity may include one or more disruptive behaviors associated with a mental state. The disruptive behaviors associated with a mental state may be, for example, varying levels of agitation, distress, and/or confusion associated with the mental state. In particular, the disruptive behaviors may be associated with delirium. In some embodiments, each of the varying levels of agitation, distress, and/or confusion may be associated with a measured Richmond Agitation Sedation Score and/or a Delirium Score. It should be noted that the Richmond Agitation Sedation Score or the Delirium score may be entered into the computing system by a clinician as input. The system then manages behavioral change based on the measured score as input.

In some embodiments, the audible and/or visual content presented to the patient includes sounds and/or images. The images may include two-dimensional (2D) video layered with three-dimensional (3D) animations. The images may include nature-based imagery. Further, the content in the images may be synchronized to the time of day in which the images are presented to the patient.

Aspects of the invention provide methods for generating visual content. The methods include the steps of generating a first layer of real-world video on a loop; overlaying the first layer with 3D animations; and controlling the movement of the 3D animations, wherein the animations spawn, move, and/or decay based on patient-generated biometric data.

### BRIEF DESCRIPTION OF THE DRAWINGS

FIG. 1 is a block diagram illustrating one embodiment of an exemplary system for providing automated behavior monitoring and modification consistent with the present disclosure.

FIG. 2 is a block diagram illustrating the audio/visual device and sensors of FIG. 1 in greater detail.

FIG. 3 is a block diagram illustrating the computing system of FIG. 1 in greater detail, including various components of the computing system for receiving and analyzing, in real time, patient activity data captured by the sensors and, based on such analysis, dynamically adjusting a level of output of audible and/or visual content to the patient.

FIGS. 4A, 4B, 4C, and 4D are diagrams illustrating one embodiment of an algorithm run by the computing system for analyzing patient activity data, specifically analyzing input received from a microphone capturing patient vocalization, and determining a level of increase or decrease in patient vocalization over a period of time based on the analysis.

FIGS. 5A, 5B, 5C, 5D, and 5E are diagrams illustrating another embodiment of an algorithm run by the computing system for analyzing patient activity data, specifically analyzing input received from a camera capturing patient motion, and determining a level of increase or decrease in patient motion over a period of time based on the analysis.

FIG. 6 is a diagram illustrating one embodiment of an algorithm, labeled as a butterfly control algorithm, run by the computing system for generating and dynamically adjusting levels of output of at least visual content (i.e., depictions of butterfly(ies)) based, at least in part, on adjustable predefined ratios associated with the patient movement and/or vocalization analysis.

FIG. 7 is a diagram illustrating another embodiment of an algorithm, labeled as a flower control algorithm, run by the computing system for generating and dynamically adjusting levels of output of another form of visual content (i.e., depictions of flower(s)) based, at least in part, on adjustable predefined ratios associated with the patient movement and/or vocalization analysis.

FIG. 8 illustrates an embodiment of the algorithm output for control of the visual content, or scene management, presented to the patient.

FIG. 9 illustrates an exemplary embodiment of the visual content presented to a patient via a display.

FIG. 10 illustrates a method for generating visual content according to one embodiment of the invention.

FIG. 11 is an exploded view of an exemplary system consistent with the present disclosure, illustrating various components associated therewith.

FIG. 12 illustrates a back view, side view, and front view of an exemplary system consistent with the present invention.

FIG. 13 illustrates a system according to one embodiment of the invention and positioned at the foot of the bed of a patient.

FIG. 14 illustrates a flow diagram used to analyze participants in the study described in Example 1.

FIG. 15 is a graph showing mean agitation scores from patients using the platform of the present invention compared to a control set in a research clinical trial for studying the level of agitation in agitated delirious patients compared to standard care alone.

FIG. 16 is a graph showing agitation reduction in patients receiving PRN (pro re nata) medications upon intervention with systems of the invention.

### DETAILED DESCRIPTION

By way of overview, the present invention is directed to a system for monitoring and analyzing patient behavior and subsequently providing automated audible and/or visual content to the patient in an attempt to arrest and de-escalate disruptive or agitated behaviors in the patient.

Aspects of the invention may be accomplished using a platform (i.e. system) configured to receive and analyze patient input and, based on such analysis, present audible and/or visual content to the patient to reduce anxiety and/or agitation in the patient. The platform may include, for example, an audio/visual device, which may include a display with speakers (i.e., a television, monitor, tablet computing device, or the like) for presenting the audible and/or visual content. The system utilizes various sensors for capturing a patient’s activity during presentation of the audible and/or visual content to the patient. The activity that is captured may include patient motion, vocalization, as well as physiological readings. The various sensors are able to capture a wide spectrum of the patient’s behavior at a given point in time, thereby providing data points, in real time, of a patient’s distress level.

The platform further includes a computing system for communicating and exchanging data with the audio/visual device and the one or more sensors. In particular, the computing system may include, for example, a local or remote computer comprising one or more processors (a single or multi-core processor(s), digital signal processor, microcontroller, or other processor or processing/controlling circuit) coupled to non-transitory, computer-readable memory containing instructions executable by the processor(s) to cause the computing system to follow a process in accordance with the disclosed principles, etc.

In particular, patient activity data is received by the computing system and analyzed based on monitoring and evaluation algorithms. Upon performing analysis of the patient activity data, the computing system is able to generate and vary the output of content (i.e., audible and/or visual content) by continuously applying content generation algorithms to the analyzed data. In this way, based on the analysis of captured patient activity data, the system is able to dynamically control output of audible and/or visual content as a means of distracting and/or engaging the patient so as to ultimately deescalate a patient’s distress level. As the anxious and aggressive behaviors calm, so too does the output, reducing the agitation levels of the patient and making the patient more receptive to care. The system is designed to integrate into the patient care pathway with minimal training and without the need for one-on-one attendance.

For the sake of clarity and ease of description, the systems described herein and audible and/or visual content provided by such systems may be provided as a means of treating patients exhibiting disruptive behavior associated with delirium. However, it should be noted that systems of the present invention may be used for modulating any disruptive behavior associated with other mental states, particularly those related to nervous system diseases, neurocognitive disorders, and/or mental disorders.

As noted above, delirium is a fluctuating state of confusion and agitation that affects between 30-60% of acute care patients annually and as many as 80% of critical care patients. Delirium is rapid in its onset and may persist for as little as hours or as long as multiple weeks. It is categorized into three types; hypoactive, hyperactive and mixed where patients can fluctuate between states. Hyperactive delirium, while less prevalent, attracts significant clinical attention and resources due to associated psychomotor agitation which complicates care. Patients may experience hallucinations or delusions and become aggressive or combative, posing a risk of physical harm to themselves and healthcare staff. Delirium has wide reaching implications in terms of financial cost to the healthcare system. It has been quoted as costing the US healthcare system in the region of 38-152 billion dollars annually in extended hospital stays, resource allocation and reliance on pharmacological therapies. Delirium complicates the provision of care by healthcare staff, and has been linked to increased risk of death and poor overall outcomes.

Some of the difficulty in identifying effective strategies for delirium is the multitude of precipitating or contributing factors that may lead to its development. Those with underlying brain health issues such as dementia are already at a predisposed risk of developing the condition. Imbalances in electrolytes, polypharmacy, sleep disturbance, underlying disease process and/or surgical intervention, all commonplace in the critical care population, are considered risk factors. Due to its multifactorial nature, it is important to examine and correct the underlying disease etiology wherever possible.

Standard care is still largely reliant on chemical and physical restraints. Pharmacological agents can carry significant risk and side effects and there is little proof that any singular non-pharmacological intervention is successful in mitigating delirium. A multi-modal approach to management that encompasses not just delirium but also pain, agitation, immobility and sleep disturbance is now recommended in the current PADIS (Pain agitation delirium immobility and sleep disruption) clinical guidelines for Intensive Care Unit (ICU) patients and the ICU Liberation Bundle. This includes the maintenance of sleep cycles, early mobilization, frequent reorientation and measures to reduce pain, anxiety and agitation. However, recommendations for management, using non-pharmacological interventions, are made on the basis of generally poor-quality studies. The 2018 PADIS guidelines rate the majority of recommendations as having low or very low quality of evidence. The present invention addresses the need for effective strategies to ensure the best possible outcomes for patients.

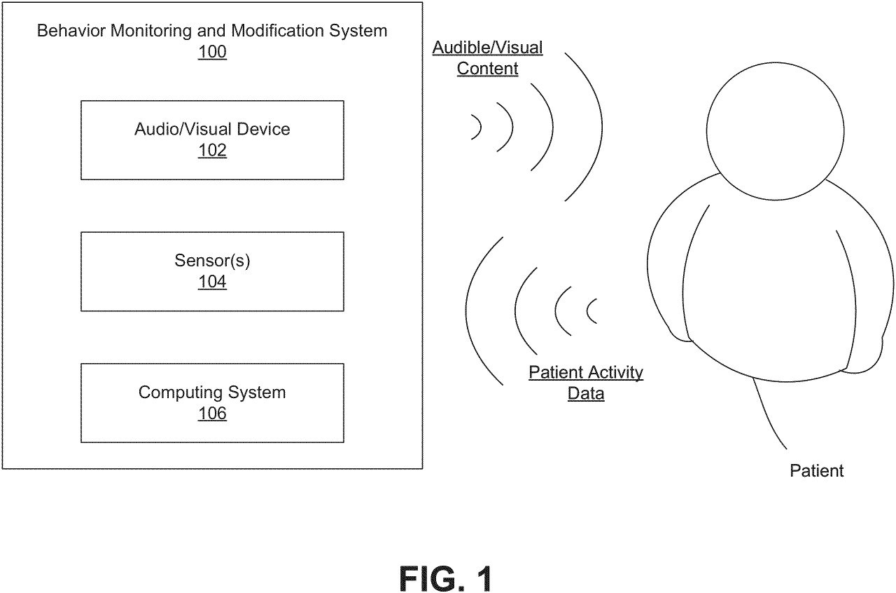

FIG. 1 is a block diagram illustrating one embodiment of an exemplary system 100 for providing automated behavior monitoring and modification consistent with the present disclosure. The behavior monitoring and modification system 100 includes an audio/visual device 102, one or more sensors 104, and a computing system 106 communicatively coupled to one another (i.e., configured to exchange data with one another).

The audio/visual device 102 is configured to present audible and/or visual content to a patient exhibiting one or more disruptive behaviors associated with a mental state. The disruptive behaviors associated with a mental state may include, but are not limited to, physical aggression towards others, threats of violence or other verbal aggression, agitation, unyielding argument or debate, yelling, or other forms of belligerent behaviour that may threaten the health and safety of the patient and healthcare providers. The one or more disruptive behaviors may be varying levels of agitation, distress, and/or confusion associated with the mental state.

In one embodiment, the mental state may be delirium associated with, for example, nervous system diseases, neurocognitive disorders, or other mental disorders. Delirium is an abrupt change in the brain that causes mental confusion and emotional disruption. Delirium is a serious disturbance in mental abilities that results in confused thinking and reduced awareness of the environment. The start of delirium is usually rapid, within hours or a few days. Elderly persons, persons with numerous health conditions, or people who have had surgery are at an increased risk of delirium.

As previously described, the mental state may be related to other medical conditions. For example, the patient may be a child or adolescent with a Disruptive Behavior Disorder. The patient may be an elderly or other person in long-term care, hospice or a hospital situation. In some instances, the patient may have Post-Traumatic Stress Disorder (PTSD). In some embodiments, the patient may be a prisoner. These non-limiting examples are meant to illustrate that systems of the invention are applicable to provide automated behavior monitoring and modification for any patient exhibiting disruptive behaviors associated with a mental state. In one embodiment, systems of the present invention provide automated behavior monitoring and modification for a hospitalized adult experiencing hyperactive delirium. In this embodiment the system functions as an interactive behaviour modification platform to arrest and de-escalate agitated behaviors in the hospitalized elderly experiencing hyperactive delirium.

As an overview, systems and methods of the invention provide a novel digital interactive behavior modification platform. The display produces nature imagery, for example a virtual garden, in response to patient movement and vocalization. Accordingly, the system may be used, for example, to reduce anxiety and psychomotor agitation in the hyperactive delirious critical care population. The system reduces reliance on unscheduled medication administration. In non-limiting examples, the platform provides for variations in visual content, incorporation of sound output to block disruptive and potentially distressing sounds and alarms, bio-feedback mechanisms with wearable sensors, and dose dependent responses.

The system is directed to a broad range of target populations, and offers various modalities for use including wearable and non-wearable options. For example, significant considerations must be made when assessing the feasibility of therapies within, for example, the hyperactive delirious critical care population. Wearable equipment may cause agitation or heightened anxiety in those suffering with altered cognition. Loss of perception of the surrounding environment, discomfort of the equipment or feelings of claustrophobia amongst some patients is possible. Patients in critical care often have significant amounts of equipment already attached making it difficult to then place more equipment on the patient especially if bed bound, or with injuries and dressings. Patient positioning must also be considered, as side positioning, important for reduction of pressure areas and potentially a requirement with certain injuries, is likely unattainable during periods of equipment use. Placing a headset or earphones on a patient in an anxious state may be possible, but with significantly restless or agitated patients keeping a headset and/or earphones in place challenging.

Notably, in example applications, the platform may be placed near or at the foot end of the bed or in sight of a patient should the patient be in a chair. The screen may be placed such that visualization by the patient is possible, but out of physical reach of the patient to ensure that damage or harm to the patient or damage of the equipment is not possible from grabbing or kicking the device unit by the patient. The system may be configured as a mobile device on a wheeled frame with an articulating arm, such that the position may be adjusted to ensure it is maintained within the patient’s field of vision at all times. The frame may have locking wheels and may include an inbuilt battery to minimize trip hazards and reduce the need for repositioning of equipment to allow access to power points. The device may be placed in standby mode for defined time intervals to allow for the provision of care such as mobilization, bathing or turning that requires physical interaction with the patient. The system may include an inbuilt timer operable to automatically restart the system at a defined time period. The stand-by feature may be activated multiple times as required to complete care. The on-screen experience can adjust the level of brightness according to the time of day to promote natural circadian rhythm.

As described in more detail herein, the system is responsive to changes in physical activity and vocalization for the delivery of visual content. The system uses input from, for example, a mounted camera to measure movement and sound generation as markers of agitation. This input drives the on-screen content delivery using proprietary algorithms in direct response to the level of measured agitation.

The one or more sensors 104 are configured to capture a patient’s activity during presentation of the audible and/or visual content to the patient. The activity that is captured may include patient motion, vocalization, as well as physiological readings. The various sensors are able to capture a wide spectrum of the patient’s behavior at a given point in time, thereby providing data points, in real time, of a patient’s distress level.

The computing system 106 is configured to receive and analyze the patient activity data captured by the one or more sensors 104. As described in greater detail herein, the computing system 106 is configured to receive and analyze, in real time, patient activity data and determine a level of increase or decrease in patient activity over a period of time. In turn, the computing system 106 is configured to dynamically adjust a level of output of the audible and/or visual content from the audio/visual device 106 to correspond to the determined level of increase or decrease in patient activity. In this way, based on the analysis of captured patient activity data, the computing system 106 is able to dynamically control output of audible and/or visual content as a means of distracting and/or engaging the patient so as to ultimately deescalate a patient’s distress level. Accordingly, systems of the invention use proprietary algorithms to compute the average movement and vocalization input over a defined time interval, for example every two seconds, and then dynamically adjusts the level of on-screen content when a significant fluctuation in movement and/or vocalization has occurred as compared to the previous interval.

The behavior monitoring and modification system 100 may be incorporated directly into the institutional/medical setting in which the patient resides, such as within an emergency room, critical care, or hospice care setting. For example, the behavior monitoring and modification system 100 may be provided as an assembled unit (i.e., multiple components provided either on a mobile cart or other carrier, or built into the construct of the setting). Yet still, in some embodiments, the behavior monitoring and modification system 106 may be provided as a single unit (i.e., a single computing device in which the audio/visual device 102, sensors 104, and computing system 106 are incorporated into a single device, such as a tablet, smart device, or virtual reality headset). Yet still, in other embodiments, the system 100 may be combination of the above components.

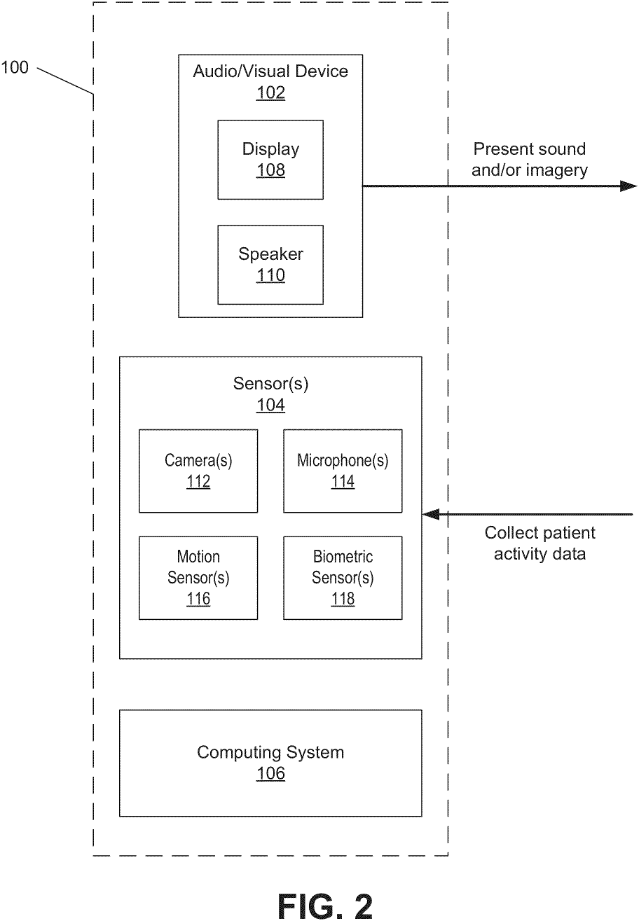

FIG. 2 is a block diagram illustrating the audio/visual device 102 and sensors 104 of FIG. 1 in greater detail. The audio/visual device 102 is configured to present audible and/or visual content to a patient exhibiting one or more disruptive behaviors associated with a mental state. The audio/visual device may include a display 108 and one or more speakers 110. The display 108 may be integrated into the system or as a stand-alone component. For example, the audio/visual device 102 may be associated with a computer monitor, television screen, smartphone, laptop, and/or tablet. The speakers 110 may be integrated into the audio/visual device, may be connected via a hard-wired connection, or may be wirelessly connected as is known in the art.

During presentation of audible and/or visual content to the patient, the one or more sensors 104 are configured to continuously capture patient activity data. The patient activity may include at least one of patient motion, vocalization, and physiological parameters/characteristics.

In some embodiments, capturing the patient’s activity data via sensor measurements may be generated at defined intervals, for example approximately every 2 seconds, throughout the active period. Each session may have a unique identifier and may also be recognizable through date and time stamps. For patients who are mechanically ventilated, the microphone function may be disabled to avoid auditory activation by the ventilator. In some embodiments, measurement generates activity logs within the system represented numerically in tabular form, for example as shown in Table 1A.

TABLE 1A

example of activity logs
(Part 1)

| _id | sessionId | sensorType | sensorValue |
|---|---|---|---|
| 60427b2077f32877e8fdb04f | 60427b1e77f32877e8fdb04e | MOVEMENT | 0.024789 |
| 60427b2077f32877e8fdb050 | 60427b1e77f32877e8fdb04e | VOLUME | 0.000702 |
| 60427b2277f32877e8fdb051 | 60427b1e77f32877e8fdb04e | VOLUME | 0.000349 |
| 60427b2277f32877e8fdb052 | 60427b1e77f32877e8fdb04e | MOVEMENT | 0.010558 |
| 60427b2477f32877e8fdb053 | 60427b1e77f32877e8fdb04e | MOVEMENT | 0.031311 |
| 60427b2477f32877e8fdb054 | 60427b1e77f32877e8fdb04e | VOLUME | 0.00055 |
| 60427b2677f32877e8fdb055 | 60427b1e77f32877e8fdb04e | VOLUME | 0.006116 |
| 60427b2677f32877e8fsb056 | 60427b1e77f32877e8fdb04e | MOVEMENT | 0.036473 |
| 60427b2877f32877e8fdb057 | 60427b1e77f32877e8fdb04e | VOLUME | 0.054868 |
| 60427b2877f32877e8fdb058 | 60427b1e77f32877e8fdb04e | MOVEMENT | 0.005466 |
| 60427b2a77f32877e8fdb059 | 60427b1e77f32877e8fdb04e | MOVEMENT | 0.003432 |
| 60427b2a77f32877e8fdb05a | 60427b1e77f32877e8fdb04e | VOLUME | 0.044183 |
| 60427b2c77f32877e8fdb05b | 60427b1e77f32877e8fdb04e | VOLUME | 0.036366 |
| 60427b2c77f32877e8fdb05c | 60427b1e77f32877e8fdb04e | MOVEMENT | 0.011468 |
| (Part 2) | | | |
| phase | createdAt | | |
| EXPERIENCE | Fri Mar. 05, 2021 10:40:32 GMT-0800 (Pacific Standard Time) | | |
| EXPERIENCE | Fri Mar. 05, 2021 10:40:32 GMT-0800 (Pacific Standard Time) | | |
| EXPERIENCE | Fri Mar. 05, 2021 10:40:34 GMT-0800 (Pacific Standard Time) | | |
| EXPERIENCE | Fri Mar. 05, 2021 10:40:34 GMT-0800 (Pacific Standard Time) | | |
| EXPERIENCE | Fri Mar. 05, 2021 10:40:36 GMT-0800 (Pacific Standard Time) | | |
| EXPERIENCE | Fri Mar. 05, 2021 10:40:36 GMT-0800 (Pacific Standard Time) | | |
| EXPERIENCE | Fri Mar. 05, 2021 10:40:38 GMT-0800 (Pacific Standard Time) | | |
| EXPERIENCE | Fri Mar. 05, 2021 10:40:38 GMT-0800 (Pacific Standard Time) | | |
| EXPERIENCE | Fri Mar. 05, 2021 10:40:40 GMT-0800 (Pacific Standard Time) | | |
| EXPERIENCE | Fri Mar. 05, 2021 10:40:40 GMT-0800 (Pacific Standard Time) | | |
| EXPERIENCE | Fri Mar. 05, 2021 10:40:42 GMT-0800 (Pacific Standard Time) | | |
| EXPERIENCE | Fri Mar. 05, 2021 10:40:42 GMT-0800 (Pacific Standard Time) | | |
| EXPERIENCE | Fri Mar. 05, 2021 10:40:44 GMT-0800 (Pacific Standard Time) | | |
| EXPERIENCE | Fri Mar. 05, 2021 10:40:44 GMT-0800 (Pacific Standard Time) | | |

For example, the sensors 104 may include camera(s) 112, microphone(s) 114, motion sensor(s) 116, and biometric sensor(s) 118. As such, the camera 112 may be used to capture images of the patient, in which such images may be used to determine a patient’s motion, such as head movement, body movement, physical gestures, and/or facial expressions which may be indicative of a level of agitation or disruptive behavior. The motion sensor(s) 116 may also be useful in capturing motion data associated with a patient’s motion (i.e., body movement and the like).

The microphone(s) 114 may be used to capture audio data associated with a patient vocalization, which may include specific words and/or utterances, as well as corresponding volume or tone of such words and/or utterances.

The biometric sensor(s) 118 may be used to capture physiological readings of the patient. In particular, the biometric sensor(s) 118 may be used to collect measurable biological characteristics, or biometric signals, from the patient. Biometrics signals may include, for example, body measurements and calculations related to human characteristics. These signals, or identifiers, are the distinctive, measurable characteristics used to label and describe individuals, often categorized as physiological characteristics. The biometric signals may also be behavioral characteristics related to the pattern of behavior of the patient. In some embodiments, the biometric sensor(s) 118 may be used to collect certain physiological readings, including, but not limited to, a patient’s blood pressure, heart rate, heart rate variability, temperature, respiratory rate and depth, skin conductance, and oxygen saturation. Accordingly, the sensors 118 may include sensors commonly used in the measuring a patient’s vital signs and capable of capturing patient activity data as is known to persons skilled in the art.

As described in more detail below, the sensors 104 are operably coupled with the computing system 106 to thereby transfer the captured patient activity data to the computing system 106 for analysis. In some instances, the sensors 104 may be configured to automatically transfer the data to the computing system 106. In other embodiments, data from the sensors 104 may be manually entered into the system by, for example, a healthcare provider or the like.

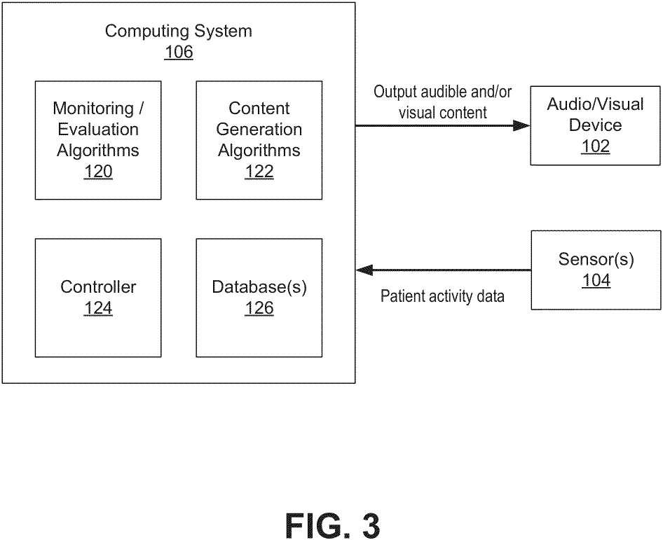

FIG. 3 is a block diagram illustrating the computing system 106 of FIG. 1 in greater detail. The computing system 106 is configured to receive and analyze the patient activity data received from the sensors 104 and, in turn, generate audible and/or visual content to be presented to the patient, via the audio/visual device 102 based on such analysis. More specifically, the computing system 106 is configured to receive and analyze, in real time, patient activity data from the one or more sensors 104 and determine a level of increase or decrease in patient activity over a period of time. The computing system 106 is configured to dynamically adjust the level of output of the audible and/or visual content from the audio/visual device 102 to correspond to the level of increase or decrease in patient activity.

The computing system 106 may generally include a controller 124, a central processing unit (CPU), storage, and some form of input (i.e., a keyboard, knobs, scroll wheels, touchscreen, or the like) with which an operator can interact so as to operate the computing system, including making manual entries of patient activity data, adjusting content threshold levels or type, and performing other tasks. The input may be in the form of a user interface or control panel with, for example, a touchscreen. The controller 124 manages and directs the flow of data between the computing system, and the sensors, and the computing system and the audio/visual device. During operation, the computing system receives the patient activity data as input into the monitoring/evaluation algorithms. As described in greater detail herein data may be continuously and automatically received and analyzed such that the content generation algorithm dynamically adjusts the audible and/or visual content as output to the audio/visual device.

The system may include a personal and/or portable computing device, such as a smartphone, tablet, laptop computer, or the like. In some embodiments, the computing system 106 may be configured to communicate with a user operator via an associated smartphone or tablet. In the present context, the user may include a clinician, such as a physician, physician’s assistant, nurse, or other healthcare provider or medical professional using the system for behavior monitoring and modification in a patient. In some embodiments, the computing system is directly connected to the one or more sensors and the audio/visual device in a local configuration. Alternatively, the computing system may be configured to communicate with and exchange data with the one or more sensors 104 and/or the audio/visual device 102, for example, over a network.

The network may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the one or more sensors, the computing system, and the audio/visual device may be, in whole or in part, a wired connection.

The network may be any network that carries data. Non-limiting examples of suitable networks that may be used as network include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G), fifth generation (5G), and future generations of cellular-based data communication technologies, Bluetooth radio, Near Field Communication (NFC), the most recently published versions of IEEE 802.11 transmission protocol standards, other networks capable of carrying data, and combinations thereof.

In some embodiments, the network may be chosen from the internet, at least one wireless network, at least one cellular telephone network, and combinations thereof. As such, the network may include any number of additional devices, such as additional computers, routers, and switches, to facilitate communications. In some embodiments, the network may be or include a single network, and in other embodiments the network may be or include a collection of networks.

As shown, the computing system 106 may process patient activity data based, at least in part, on monitoring/evaluation and content generation algorithms 120, 122, respectively. The monitoring/evaluating algorithms 120 may be used in the analysis of patient activity data from the sensors 104. Input and analysis may occur in real time. For example, the transfer of patient activity data from the one or more sensors 104 to the computing system 106 may occur automatically or may be manually entered into the computing system 106. In turn, the computing system 106 is configured to analyze the patient activity data based on monitoring/evaluation algorithms 120. In particular, the computing system 106 may be configured to analyze data captured by at least one of the sensors 104 and determine at least a level of increase or decrease in patient activity over a period of time based on the analysis.

For example, the monitoring/evaluation algorithms 120 may include custom, proprietary, known and/or after-developed statistical analysis code (or instruction sets), hardware, and/or firmware that are generally well-defined and operable to receive two or more sets of data and identify, at least to a certain extent, a level of correlation and thereby associate the sets of data with one another based on the level of correlation.

As described in detail herein, in some embodiments, the monitoring/evaluation algorithms 120 may be used for analyzing patient activity data, specifically analyzing input received from a microphone capturing patient vocalization, and determining a level of increase or decrease in patient vocalization over a period of time based on the analysis. Volume may be calculated by finding the highest level of sound, converted to a decimal percentage between 0 and 1 (0 being the lowest level and 1 the highest).

As described in detail herein, in another embodiment, the monitoring/evaluation algorithms 120 may be used for analyzing patient activity data, specifically analyzing input received from a camera capturing patient motion, and determining a level of increase or decrease in patient motion over a period of time based on the analysis. Movement may be calculated by comparing the difference in pixel density from the previous frame to the current one. The resulting value may then be averaged over the collected frames and returned as a decimal percentage of change, called the Movement Count Average. Values are between 0 and 1 with 0 showing the lowest amount of activity and one the highest

In another embodiment, the monitoring/evaluation algorithms 120 may be used for analyzing patient activity data, specifically analyzing input received from the biometric sensors capturing physiological readings, and determining a level of increase or decrease in the patient’s physiological readings over a period of time based on the analysis.

It should be noted that the analyzed patent activity data, including the determined level of patient activity, may generally be associated with levels of disruptive behavior, such as agitation, distress, and/or confusion associated with a mental state. For example, varying levels of agitation, distress, and/or confusion may be associated with a measured Richmond Agitation Sedation Score and/or a Delirium Score.

The Richmond Agitation Sedation Scale (RASS) is an instrument developed by a team of critical care physicians, nurses, and pharmacists to assess the level of alertness and agitated behavior in critically-ill patients. The RASS is a 10-point scale ranging from -5 to +4, with levels +1 to +4 describing increasing levels of agitation. Level +4 is combative and violent, presenting a danger to staff. In some embodiments, the RASS score of a patient is entered into the system at regular defined or undefined intervals. The RASS score may be entered manually by healthcare staff.

In some embodiments, the Delirium Score may be calculated by the computing system based on one or more of delirium stratification scales, for example, the Delirium Detection Score (DDS), the Cognitive Test of Delirium (CTD), the Memorial Delirium Assessment Scale (MDAS), the Intensive Care Delirium Screening Checklist (ICDSC), the Neelon and Champagne Confusion Scale (NEECHAM), or the Delirium Rating Scale-Revised-98 (DRS-R-98). The Delirium Score may be automatically entered into the system or may be manually entered by healthcare providers. As with other sensor data, the Delirium Score may be continually refreshed and entered into the system in real time.

In some embodiments, the system includes using video data collected from one or more sessions to use for blinded assessment of agitation scoring by trained personnel. Scoring may be based on the standardize Richmond Agitation Sedation Score tool, and correlated with the patient activity score, for example, movement count average and sound input scores, computed by the system algorithms and stored in the system patient/session logs.

The computing system 106 then applies content generation algorithms 122 so as to vary the output of audible and/or visual content from the audio/visual device 102 based on changing patient input received and analyzed based on the monitoring/evaluating algorithms 120. As discussed in more detail below, the content generation algorithm generates and/or adjusts the output of content, specifically audible and/or visual content. As described in detail herein, visual content is primarily image-based and may include images (static and/or moving), videos, shapes, animations, or other visual content. For example, the visual content may be nature-based imagery comprising, for example, flowers, butterflies, a water scene, and/or beach scene. The visual content may comprise alternate visual content options. In some embodiments, the system may provide for a choice of patient and/or substitute decision-maker selected visual content. In some embodiments, the choice of visual content may be randomized.

Visual content used with systems and methods of the invention is precisely created using methods disclosed herein. For example, a first or base layer of actual nature video on a loop may be used to ground the visual experience. The first layer is intended to calm and/or lull the patient by remaining constant. This is in contrast to nature videos or TV, as there are no sudden changes in the pixels of this layer that subliminally confuse the mind. Thus, the layer is a constant grounding state.

Bespoke three-dimensional (3D) animations may then overlay this base layer. As described herein, it is these illustrations, overlaid upon the base layer, that spawn, move, and decay based on the patient-generated biometric data. For example, in non-limiting examples, the overlay may be butterflies or illustrated flowers, and may spawn, move, and/or decay based on voice agitation levels, moment, heart rate variability, and/or blood pressure.

As disclosed herein, systems of the invention generate content based on one or more of a multitude of patient-generated biometric data, including, in non-limiting examples, respiration (rate, depth) heart rate, heart rate variability, blood oxygen saturation, blood pressure, EEG, and fMRI. For example, in some embodiments, the movement and/or speed of the 3D animations may be matched to what may be an aspirational breathing pattern for a patient, for example for a person over age 65, or within the range of the breathing pattern. The 3D animations may follow a predetermined pattern. Further, the speed of the 3D animations may be controlled via the control panel.

The speed and movement of the first/video layer and the 3D animation layer may be independent, with only the speed and/or movement of the 3D animations driven by patient biometric data.

Audible content may include, for example, sounds (i.e., sound effects and the like), music, spoken word or voice content, and the like. In some embodiments, the content generation algorithm 122 is used to generate output that includes nature-based visual content and noise-cancelling or noise-masking sounds. In some embodiments, the system may include sound output. For example, the sound output frequency may be selected at a frequency that enhances the calming and anxiety-reducing effect of the visual platform. For example, the sound output may be emitted at a frequency of around 528 Hz. The sound output may comprise white noise. The inclusion of sound output as white noise may be calming and help mask or cancel out the surrounding noises of the patient care environment.

The computing system 106 may further include one or more databases 126 with which the monitoring/evaluation algorithms 120 and the content generation algorithms 122 communicate. In some embodiments, the database may include a bespoke content library for generating personalized and compelling content that captures and retains the attention of the patient and is effective for arresting and de-escalating disruptive and/or agitated behavior.

The invention may use data collected from one or more sensors, such as biosensors, as input for developing visual content. For example, the use of bio-feedback sensors may be incorporated to drive development of on-screen content by using metrics such as heart rate, heart rate variability, and respiratory rate. In some embodiments, the system provides for the continuous collection of physiological parameter values and trends over time of, for example, heart rate, heart rate variability, respiratory rate, oxygen saturation, mean arterial pressure, and vasopressure. This data may be collected from the critical care unit central monitoring systems, and de-identified for analysis. In some embodiments, the biosensor data may be used to augment content generation algorithms with the additional patient physiological data, and determine a recommended dosage or exposure duration. The data may further be used to track patient physiological response to systems of the invention to enable comparison within a patient, for example, at different time internals, across patients, for example, by age, gender, diagnosis, procedures, delirium sub-type and severity, and between different types of system interactive visual content and audio soundscapes. In other embodiments, the data is used to provide a risk-based score on the probability of a patient developing delirium, such that the system may be used proactively in the patient care.

In some embodiments, the system provides breathwork prompts and screen-based visualization exercises. This feature may be used as a tool for healthcare providers, for example by respiratory therapists, working with patients no longer requiring ventilator support. Patient respiration data, such as inspiration/expiration volume and/or flow rate, may be collected from a digital incentive spirometer and used with the systems of the invention as an interactive visualization tool, for example as a virtual incentive spirometer. In this way, patient performance may be displayed to gamify the respiration exercises crucial to lung health and recovery after being weaned off of a respirator.

In some embodiments, the system includes eye-tracking technology to determine a level of interactivity with the platform by the participant. Thus, eye-tracking may be incorporated to determine the level of patient engagement with the systems. Data obtained from measuring the level of patient engagement allows the system to more efficiently render the on-screen interactive experience. For example, in some embodiments, the system may use eye-tracking or eye movement data to render the visual content on the area currently being viewed by the patient rather than rendering the visual content on the entire screen. Further, other areas of visual content may be rendered at a lower resolution to allow for optimizing the system for use on a lower spec CPU and GPU.

In some embodiments, the system include may include pre-recorded audio cues that interrupt the existing sound output, and state orientation cues for the patient including where they currently are, e.g. hospital name and/or city location. For example, in some embodiments, on-screen re-orientation prompts at the top of the screen may continuously display time, day of the week, year, and other relevant information for orienting the patient as to time and place. The system may include a pre-recorded audio cue that interrupts the existing sound output and states for the patient orientation cues including where they are and generic information regarding being safe, that persons around them are members of the healthcare team there to help them, and the like. These prompts may be coordinated with regular orientation prompts that are given by nursing and healthcare staff throughout the day, as orientation prompts are strongly recommended for the care of patients to both prevent and manage delirium. Audio prompts may be any length, for example, in some embodiments the audio prompts may be approximately 15-30 seconds long, and may be provided in multiple languages, e.g. Punjabi, Hindu, Cantonese, Spanish.

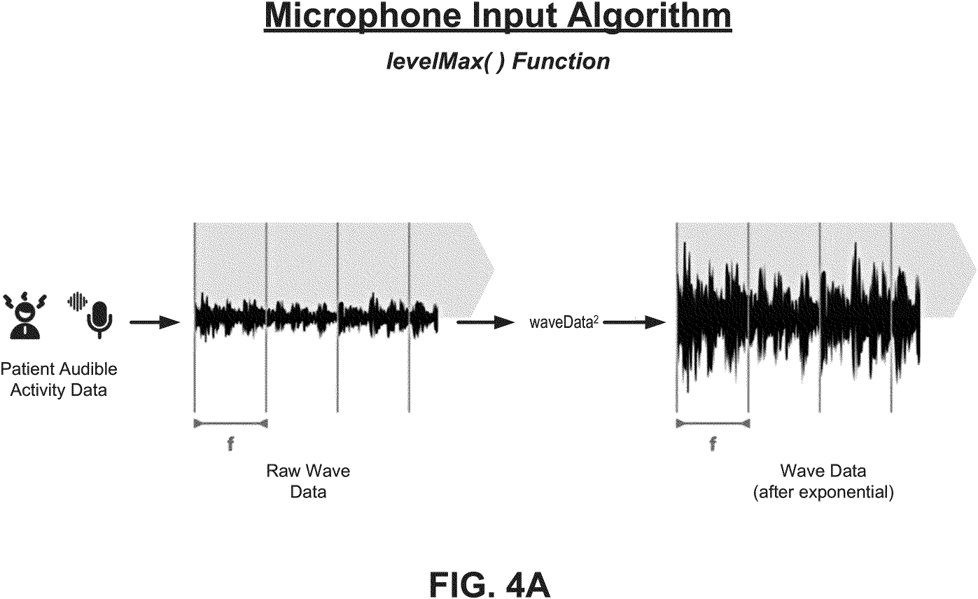

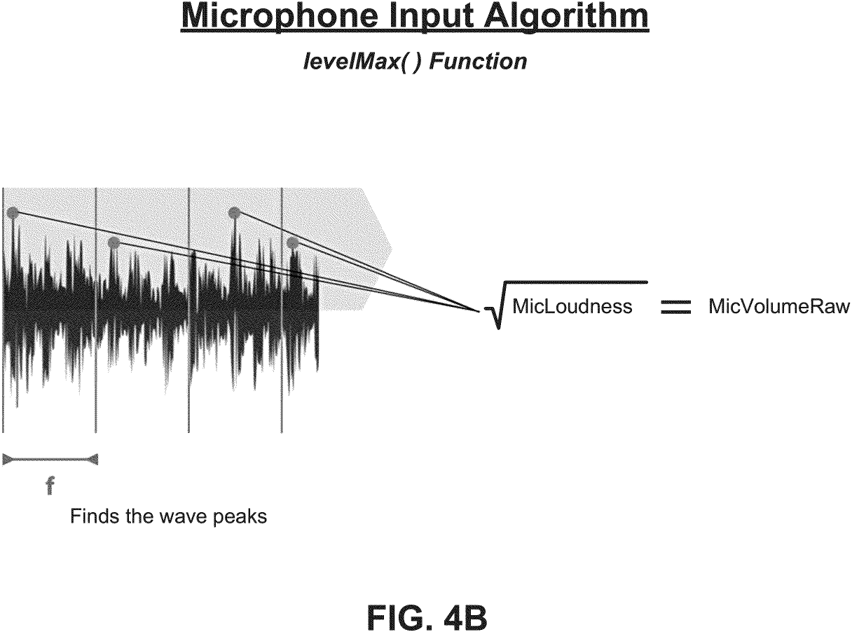

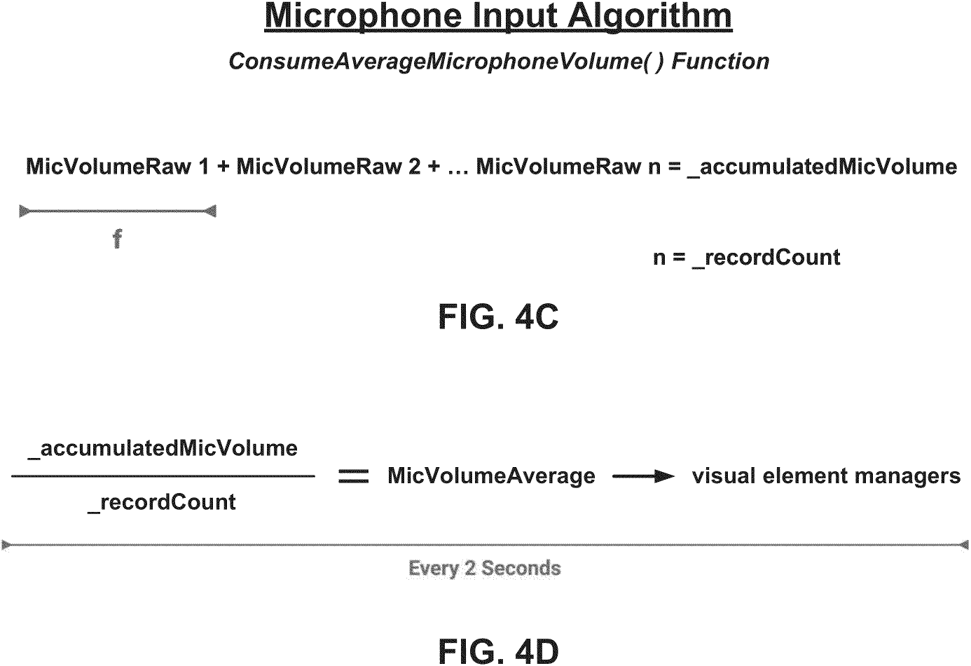

FIGS. 4A-4D illustrate one embodiment of an algorithm run by the computing system for analyzing patient activity data, specifically analyzing input received from a microphone capturing patient vocalization, and determining a level of increase or decrease in patient vocalization over a period of time based on the analysis. More specifically, the algorithm illustrated in FIGS. 4A-4D receives audible input (dB) generated by a patient (received by a microphone of the system) and converts such input into numerical data.

More specifically, and as described in greater detail herein, FIGS. 4A-4D illustrate an embodiment of a microphone input function and its use for analyzing and calculating microphone average volume, labeled as MicVolumeAverage, as an input into the content generation algorithms. The monitoring/evaluating algorithm analyzes the input via an input function. The input function analyzes the wave data received from one or more microphone sensors to calculate MicVolumeAverage that is then used by the content generation algorithm in conjunction with other inputs to generate the content output that is transferred to the audio/visual device.

In some embodiments, the system is built in a video game engine, such as Unity, for creating real-time 2D, 3D, virtual reality and augmented reality projects such as video games and animations. As such, the time references are in frames per second (fps; where f = frame), unless otherwise indicated. The fps vary depending on the workload of each frame being rendered, and the normal operating range for system is 60 fps ± 30 fps. Variations in fps are by design and undetectable by the system user(s). The input algorithm refreshes data in real time, for example every two seconds. While the actual frames per second may vary while the computing system is running, in some embodiments, the system may be optimized for sixty frames per second.

For example, FIG. 4A illustrates patient audible activity data as an input into an embodiment of the algorithm. As shown, the algorithm causes the system, in every frame, to record all of the wave peaks, (waveData) from the raw audio data and square them. The exponential function amplifies the wave signals.

As illustrated in FIG. 4B, from the amplified signals, the algorithm determines the largest results from each frame and saves the value as the current MicLoudness. To reset the amplification and return the signals to the raw data values, the square root of the MicLoudness is calculated and stored as MicVolumeRaw.

As shown in FIG. 4C, because a MicVolumeRaw data point is collected every frame, (i.e. every second), a data accumulation function is applied so as not to overburden the processor, and keep the patient experience smooth. Whenever a MicVolumeRaw data point is collected, the value is added to the variable _accumulatedMicVolume and another variable_recordCount is incremented by 1.

FIG. 4D illustrates that every two seconds the variable _accumulatedMicVolume is divided by the _recordCount to get the MicVolumeAverage used by the Visual Elements Manager (i.e. ButterflyManage.cs and FlowerManager.cs). Once MicVolumeAverage is both variables are reset to zero for the next batch of MicVolumeRaw data.

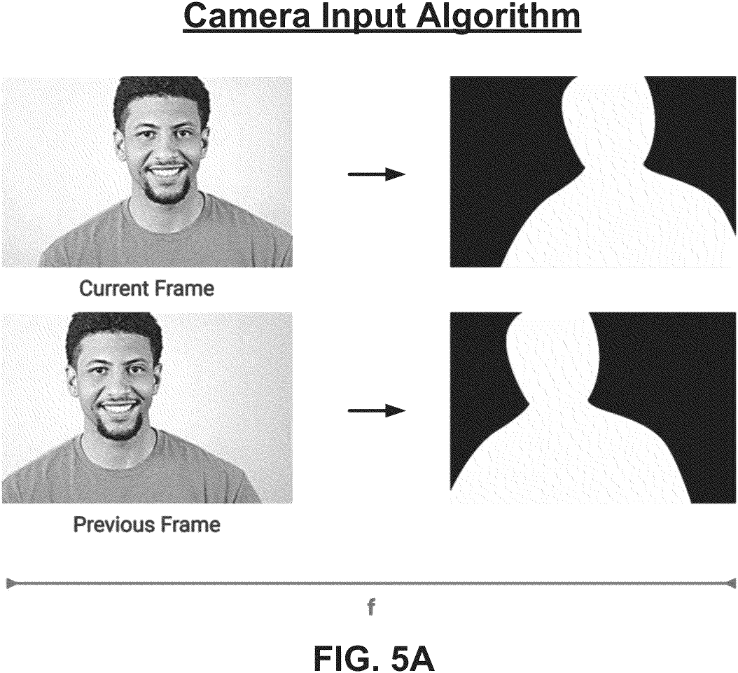

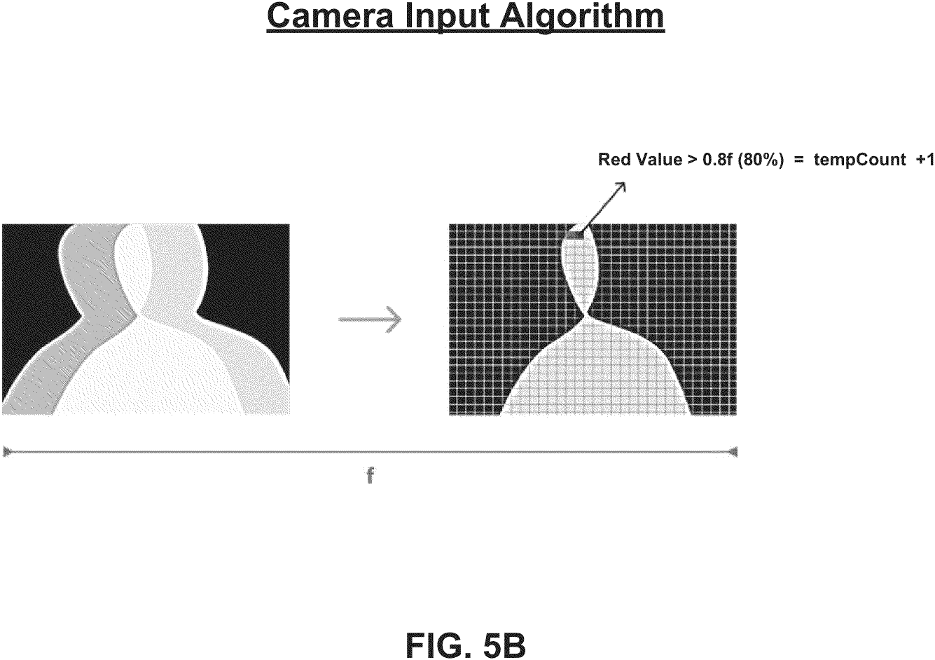

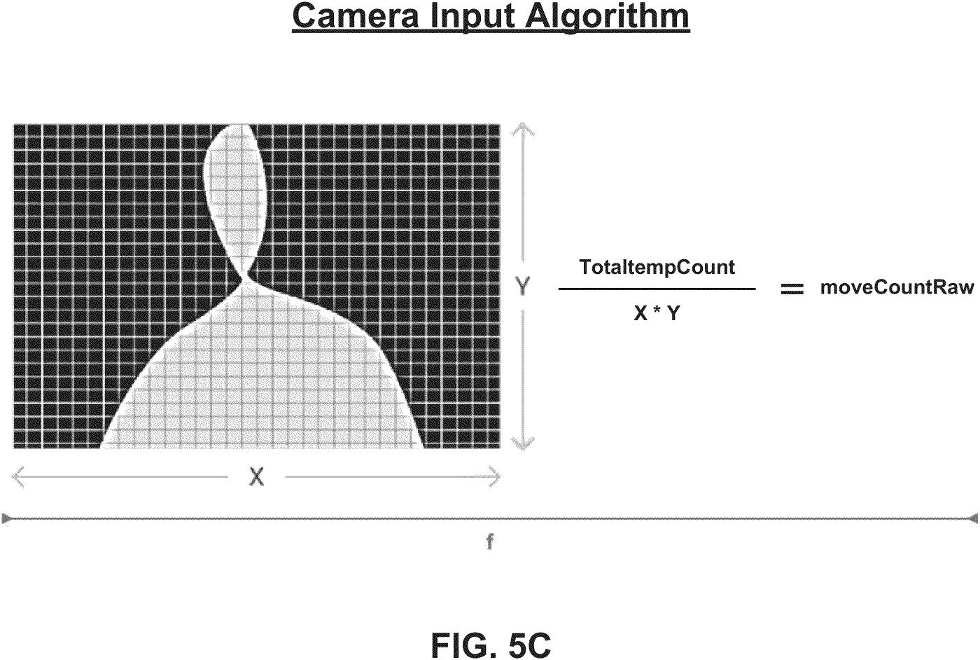

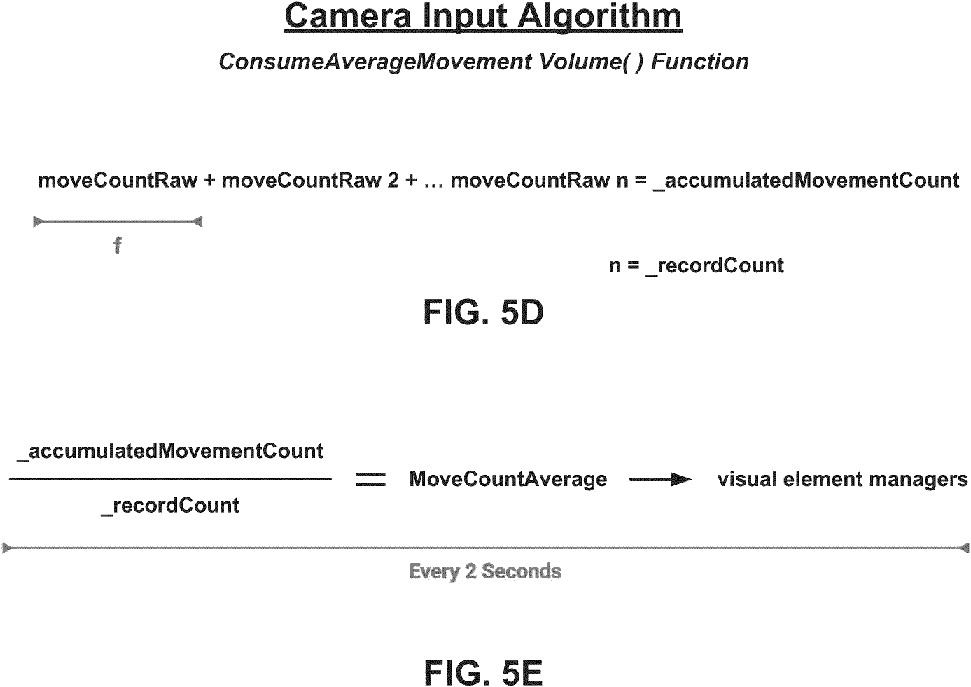

FIGS. 5A-5E illustrate another embodiment of an algorithm run by the computing system for analyzing patient activity data, specifically analyzing input received from a camera capturing patient motion, and determining a level of increase or decrease in patient motion over a period of time based on the analysis.

FIG. 5A illustrates the algorithm that takes the visual input (such as movement/motion) generated by the patient and converts it to numerical data. In some embodiments, for every frame, the system takes the current frame (image) from the webcam as well as the previous frame. An image filter is then applied to both images, making them black and white, inverting the colors, and turning up the saturation.

FIG. 5B illustrates the application of a filter to the data. Once the filter has been applied, the system compares the two frames and measures the change in every pixel. As an example, a significant change in frame difference that indicates patient movement/motion occurs when a pixel’s value is greater than or equal to 0.80 (80%), illustrated in FIG. 5B as tempCount.

FIG. 5C further illustrates that once the frame has been compared and the change in all of the pixels has been calculated, the system takes the total number of tempCount and divides it by the total number of pixels on screen. The resulting value is then stored in moveCountRaw.

As shown in FIG. 5D, because a moveCountRaw data point is collected every frame, (i.e. every second), a data accumulation function is applied so as not to overburden the processor, and to keep the patient experience seamless. Whenever a moveCountRaw data point is collected, the value is added to the variable _accumulatedMovementCount and another variable _recordCount is incremented by one.

As illustrated in FIG. 5E, every two seconds, the variable _accumulatedMovementCount is divided by the variable _recordCount to get the MoveCountAverage used by the Visual Element Managers algorithms for generating the content presented to the patient. Once MoveCountAverage is returned, both of the variables are reset to zero for the next batch of moveCountRaw data.

In some embodiments, a texture map is used as part of the analysis. The texture map may be created by placing a two-dimensional surface on the three-dimensional object, such as a patient’s face, that is being measured.

It should be noted that the microphone input function and the camera input function are intended to be non-limiting examples of the types of input functions and algorithms utilized by the system to monitor and analyze input data from the various sensors, and to generate audio and/or visual content. In some embodiments, the system may use any number of sensors, patient activity data, and input functions to monitor, analyze, and to generate the output to the audio/visual device.

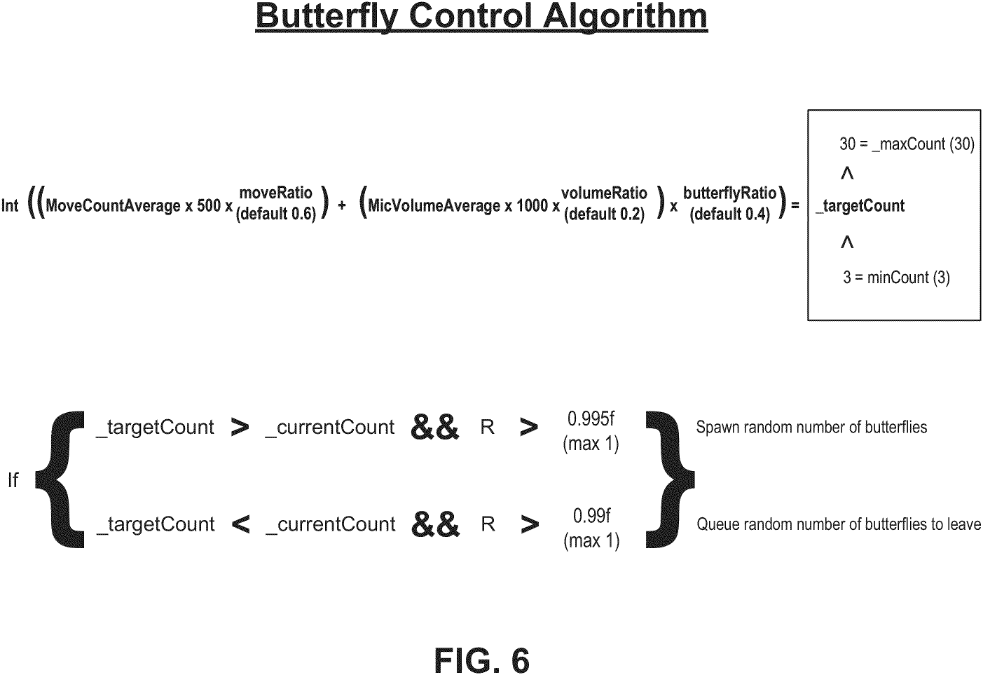

FIG. 6 is a diagram illustrating one embodiment of an algorithm run by the computing system for generating and dynamically adjusting levels of output of at least visual content (i.e., depictions of butterfly(ies)) based, at least in part, on adjustable predefined ratios associated with the patient movement and/or vocalization analysis. In some embodiments, the Butterfly Control Algorithm (ButterflyManager.cs) controls the number of butterflies present on the screen at any given time in relation to the visual and audible input produced by the patient. In the non-limiting embodiment shown in FIG. 6, the algorithm may consist of three ratios that affect the number of butterflies.

The algorithm uses MoveCountAverage and MicVolumeAverage in conjunction with predefined, adjustable ratios-labeled as moveRatio, volumeRatio, and butterflyRatio-to calculate the content generated as output to the audio/visual device. The output, labeled as _targetCount in this example, illustrates that the computing system is configured to dynamically adjust levels of output of the audible and/or visual content based on the adjustable predefined ratios. In some embodiments, randomized functions are applied to the generation and decay of audible and/or visual content to make the scene appear more natural.

As shown, the computing system 106 may use a continuously applied content generation algorithm to vary output based on changing patient activity data (i.e., changing level of patient activity). The levels of output are dynamically adjusted based on adjustable, predefined ratios applied to the patient activity data. In some embodiments, the input and output ratios driving the content generation algorithm can be optimized for different diseases, patients, and patient populations.

In other embodiments, the algorithms of the computing system are configured to dynamically adjust levels of output of the audible and/or visual content based on predefined percentage increments, which may be in the range between 1% and 100%. For example, in some embodiments, audible and/or visual content output may be increased or decreased in predefined percentage increments in the range between 5% and 50%. Yet still, in some embodiments, the predefined percentage increments may be in the range between 10% and 25%. Accordingly, in the event that a patient’s activity level increases by one, two, or more increments, or alternatively decreases by one, two, or more increments, the system may be configured to correspondingly increase or decrease the level of output of audible and/or visual content by a predefined percentage (i.e., by 5%, 10%, 25%, etc.).

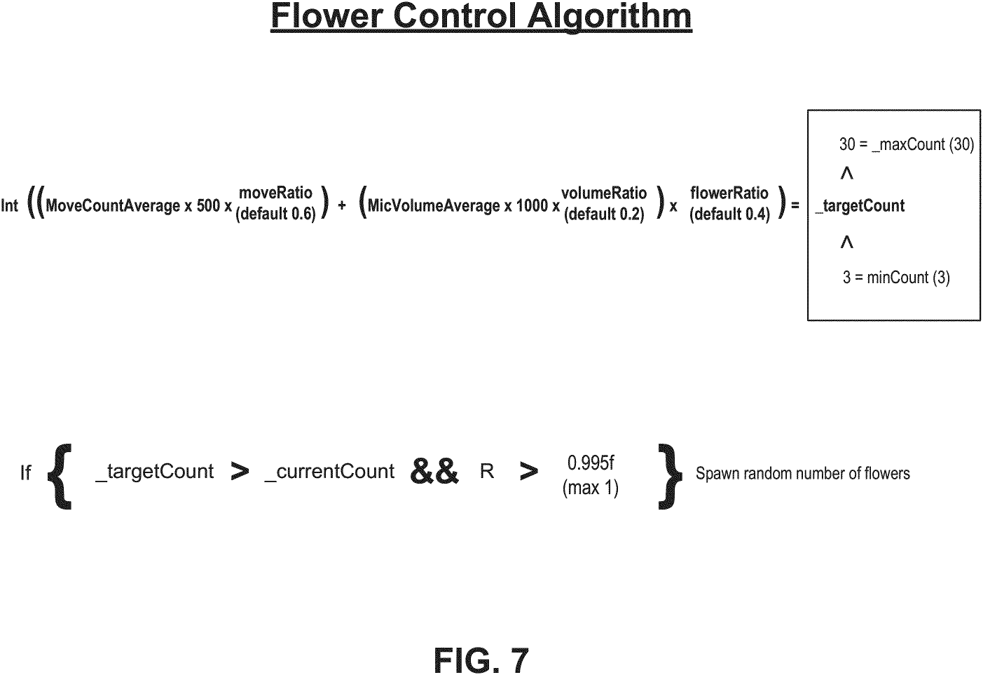

FIG. 7 is a diagram illustrating another embodiment of an algorithm run by the computing system for generating and dynamically adjusting levels of output of another form of visual content (i.e., depictions of flower(s)) based, at least in part, on adjustable predefined ratios associated with the patient movement and/or vocalization analysis. An algorithm controls the number of flowers present on the screen at any given time in relation to the visual and audible input produced by the patient. In some embodiments, the algorithm consists of three ratios that affect the number of flowers.

For example, computing system 106 may utilize the content generation algorithm 122, which utilizes MoveCountAverage and MicVolumeAverage in conjunction with predefined, adjustable ratios-labeled as moveRatio, volumeRatio, and flowerRatio-to calculate the content generated as output to the audio/visual device. The output, labeled as_targetCount in this example, illustrates that the computing system is configured to dynamically adjust levels of output of the audible and/or visual content based on these adjustable predefined ratios. The content generation algorithms illustrated herein are meant to be non-limiting examples of the types of algorithms used by the system to generate content as output for the audio/visual device.

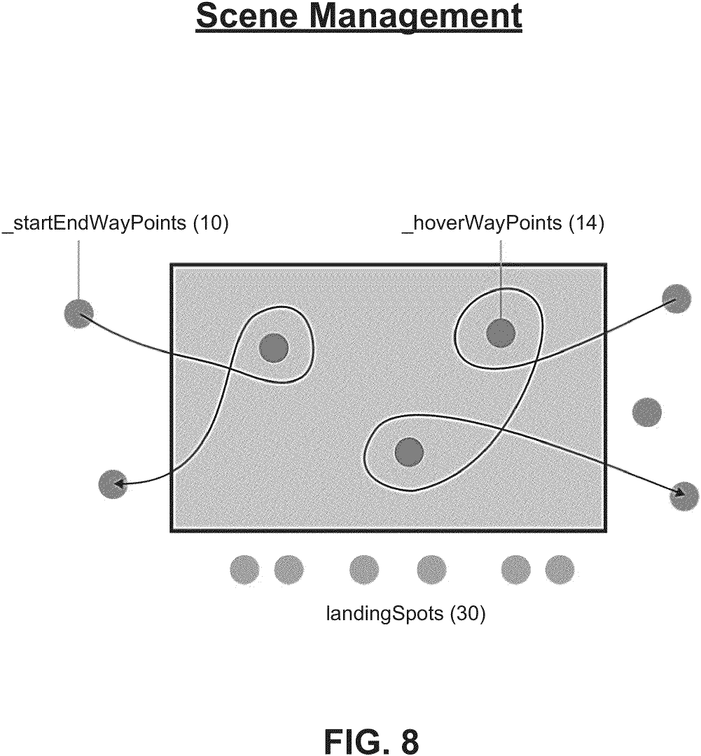

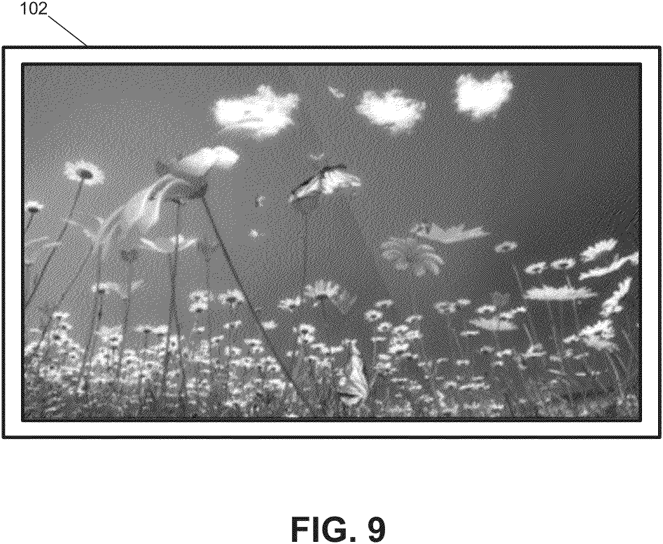

FIG. 8 shows an embodiment of an algorithm to manage the scene(s), or visual content, presented to the patient. For the scenes to appear more natural, randomized asset spawn points and hover points are used to generate and move the assets on screen. As shown in FIG. 8, as a non-limiting example, for butterflies, whenever a butterfly is queued to spawn, the algorithm randomly picks one of the 10 _startEndWayPoint to generate a butterfly. The butterfly then randomly picks one of the 14 _hoverWayPoints to move toward. Once the butterfly has reached the current hover point it randomly picks another hover point to move toward, if the butterfly has not already been queued to leave. Once queued to leave, the butterfly randomly picks a _startEndWayPoints to move toward and be despawned.

As another example, for flowers, whenever a flower is queued to spawn, the algorithm randomly picks one of, for example, 30 landingSpots to generate a flower. The flower will rise up from the landingSpots with randomized size and rotation and will stay on screen for a randomized target duration of, for example, between 15 seconds to 30 seconds. If a flower is queued to despawn prior to the pre-set duration, a flower will retract back into the landingSpots of origin and be despawned.

As shown in FIG. 9, in some embodiments, the output presented by the audio/visual device 102 to the patient may generally be in the form of nature-based patterns and nature-based imagery. FIG. 9 illustrates an exemplary embodiment of visual content presented to a patient via the audio/visual device 102. As shown, the audible and/or visual content may be nature-based imagery comprising, for example, flowers and butterflies. The audible and/or visual content may be any content related to such imagery (i.e., sounds of nature, including background noise, such as the sound of birds, wind, etc.). The content may be delivered as real 2-dimensional (2D) nature video layered with 3-dimensional (3D) animations of growing and receding flowers and butterflies in flight.

It should be noted that the visual output may be any type of imagery, and is not limited to nature-based scenery. For example, patterns, shapes, colors, waves, or the like. The visual imagery may be still or video content or a combination thereof. The video may be a sequence of images, or frames, displayed at a given frequency. The content may further be synchronized to the time of day in which the content is presented to the patient. The content may be synchronized to the time of day the images are presented to the patient. The sounds associated with the output may be noise-cancelling and/or noise-masking.

Visual content used with systems and methods of the invention is precisely created using methods disclosed herein. For example, a first or base layer of actual nature video on a loop may be used to ground the visual experience. The first layer is intended to calm and/or lull the patient by remaining constant. This is in contrast to nature videos or TV, as there are no sudden changes in the pixels of this layer that subliminally confuse the mind. Thus, the layer is a constant grounding state.

Bespoke three-dimensional (3D) animations/illustrations may then overlay this base layer. As described herein, it is these illustrations, overlaid upon the base layer, that spawn, move, and decay based on the patient-generated biometric data. For example, in non-limiting examples, the overlay may be butterflies or illustrated flowers, and may spawn, move, and/or decay based on voice agitation levels, moment, heart rate variability, and/or blood pressure.

As disclosed herein, systems of the invention generate content based on one or more of a multitude of patient-generated biometric data, including, in non-limiting examples, respiration (rate, depth) heart rate, heart rate variability, blood oxygen saturation, blood pressure, EEG, and fMRI. For example, in some embodiments, the movement and/or speed of the 3D animations may be matched to what may be an aspirational breathing pattern for a patient, for example for a person over age 65, or within the range of the breathing pattern. The 3D animations may follow a predetermined pattern. Further, the speed of the 3D animations may be controlled via the control panel.

The speed and movement of the first/video layer and the 3D animation layer may be independent, with only the speed and/or movement of the 3D animations driven by patient biometric data.

Audible content may include, for example, sounds (i.e., sound effects and the like), music, spoken word or voice content, and the like. In some embodiments, the content generation algorithm is used to generate output that includes nature-based visual content and noise-cancelling or noise-masking sounds. In some embodiments, the system may include sound output. For example, the sound output frequency may be selected at a frequency that enhances the calming and anxiety-reducing effect of the visual platform. For example, the sound output may be emitted at a frequency of around 528 Hz. The sound output may comprise white noise. The inclusion of sound output as white noise may be calming and help mask or cancel out the surrounding noises of the patient care environment.

As previously described, the computing system 106 receives and analyzes, in real time, patient activity data from the one or more sensors and determines a level of increase or decrease in patient activity over a period of time. The computing system 106 dynamically adjusts the level of output of the audible and/or visual content from the audio/visual device to correspond to the determined level of increase or decrease in patient activity.

In some embodiments, the increase in patient activity may be one or more of increased patient motion, increased vocalization, and increased levels of physiological readings as measured by the one or more sensors. As such, the computing system 106 is configured to automatically increase the level of output of audible and/or visual content to correspond to the increase in patient activity. In some embodiments, this increase in the level of output of audible and/or visual content may include, but is not limited to, an increase in an amount of visual content presented to the patient, an increase in a type of visual content presented to the patient, an increase in movement of visual content presented to the patient, an increase in a decibel level of audible content presented to the patient, an increase in frequency and/or tone of audible content presented to the patient, and an increase in tempo of audible content presented to the patient.

Likewise, in some embodiments, the decrease in patient activity comprises at least one of decreased patient motion, decreased patient vocalization, and decreased levels of patient physiological readings. As such, the computing system 106 is configured to decrease a level of output of audible and/or visual content to correspond to a decrease in patient activity. The decreased level of output of audible and/or visual content may include, but is not limited to, a decrease in an amount of visual content presented to the patient, a decrease in a type of visual content presented to the patient, a decrease in movement of visual content presented to the patient, a decrease in a decibel level of audible content presented to the patient, a decrease in frequency and/or tone of audible content presented to the patient, and a decrease in tempo of audible content presented to the patient.

Thus, the computing system 106 is configured to control the parameters of the audible and/or visual content such as the frequency, rate, and type of images and/or sounds, tone, tempo, and movement as examples.

Further, the computing system 106 may be configured to dynamically adjust levels of output of the audible and/or visual content based on adjustable predefined ratios applied to patient activity data. For example, in some embodiments, randomization functions are applied to the generation and decay of audible and/or visual content so as to make the scene appear more natural to the viewer.

Aspects of the invention include a method for creating visual content. Visual content provided in the systems and methods of the invention is precisely created for automated behavior monitoring and modification in a patient. The method includes generating a first layer of actual nature video on a loop. The first layer may move, sway, and/or flow to ground the experience. For example, the visual content may be a looped video of real coneflowers swaying in a prairie breeze. In non-limiting examples, the first layer may be actual video of sea coral and/or sea flowers waving in an ocean drift. The first layer is intended to calm and/or lull the patient by remaining constant. This is in contrast to nature videos or TV, as there are no sudden changes in the pixels of this layer that subliminally confuse the mind. Thus, the layer is a constant grounding state.

The method further comprises overlaying the base layer with bespoke three-dimensional (3D) animations and/or illustrations. It is these illustrations/animations that spawn, move, and decay based on the patient-generated biometric data. For example, in non-limiting examples, the overlay may be butterflies or illustrated flowers, and may spawn, move, and/or decay based on voice agitation levels, moment, heart rate variability, and/or blood pressure.

As disclosed herein, systems of the invention generate content based on one or more of a multitude of patient-generated biometric data, including, in non-limiting examples, respiration (rate, depth) heart rate, heart rate variability, blood oxygen saturation, blood pressure, EEG, and fMRI. For example, in some embodiments, the movement and/or speed of the 3D animations may be matched to what may be an aspirational breathing pattern for a patient, for example for a person over age 65, or within the range of the breathing pattern. The 3D animations may follow a predetermined pattern. Further, the speed of the 3D animations may be controlled via the control panel.

The speed and movement of the first/video layer and the 3D animation layer may be independent, with only the speed and/or movement of the 3D animations driven by patient biometric data.

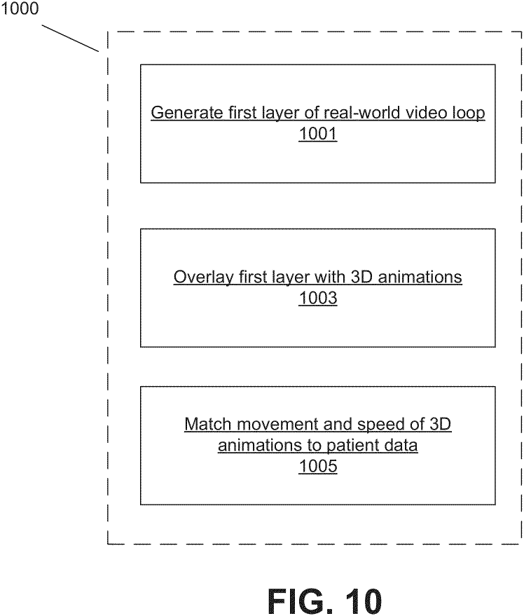

FIG. 10 illustrates a method 1000 for generating/creating visual content according to one embodiment of the invention. The method includes the steps of generating 1001 a first layer of real-world video on a loop; overlaying 1003 the first layer with bespoke animations; and controlling 1005 the movement of the 3D animations, wherein the animations spawn, move, and/or decay based on patient-generated biometric data.

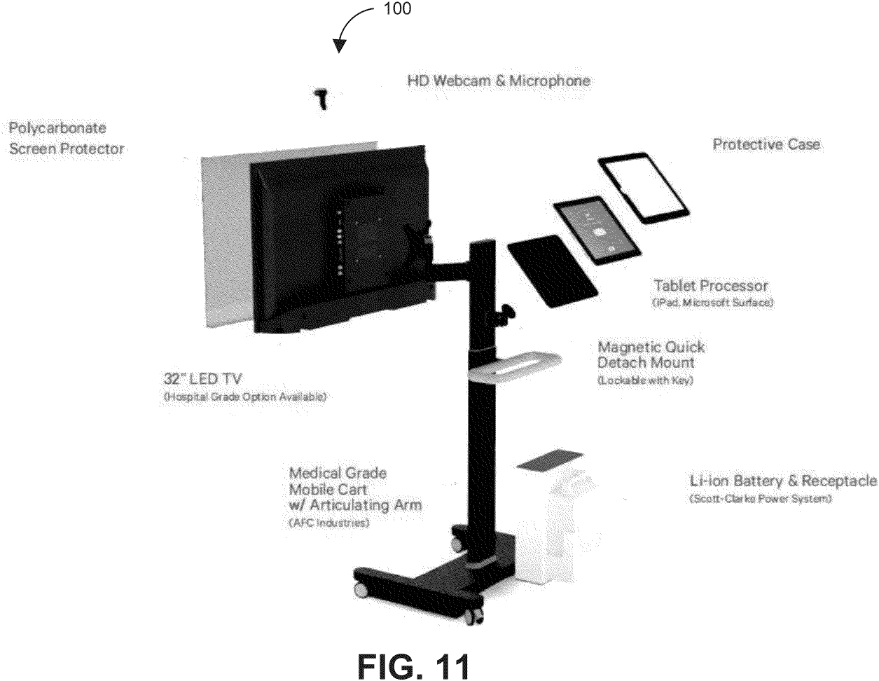

FIG. 11 is an exploded view of an exemplary system 100 consistent with the present disclosure, illustrating various components associated therewith. As shown, the system 100 may include a touchscreen control panel, for example a tablet, and a processor with controller. The tablet or control panel may include a protective case. The system 100 may be mobilized and provided on a cart. For example, the cart may be a medical grade mobile cart with an articulating arm and a handle for easily moving the cart into position. In the illustrated embodiment, the audio/visual device is an LED television attached to an articulating arm which is attached to an upright stand member of the cart. The LED television may be medical grade. The audio/visual device may include a screen protector, for example a polycarbonate screen protector. The cart may have a wheeled based for easily moving in and out of a patient’s room. The stand may include a compartment or receptacle for storing the computer processing unit and a battery docking station. As shown, the system may include a magnetic quick-detach mount, for example to secure the control panel, which may include a lockable key. The system may include a medical grade rechargeable battery so that the system can be operated as a battery-powered unit to increase mobility and provide accessibility to patients in need. The system may include a webcam mounted to the audio/visual display as an input sensor, a microphone as a second input sensor and speakers (not shown).

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### EXAMPLES

Aspects of the invention are illustrated in the examples provided below. In the study, the behavior monitoring and modification system of the invention is referred to as the MindfulGarden platform.

### Example 1: Use of a Novel Digital Intervention to Reduce Delirium-Associated Agitation: A Randomized Clinical Trial

### Introduction:

Delirium is an acute neuropsychiatric disorder of fluctuating confusion and agitation that affects as many as 80% of patients in critical care. Hyperactive delirium consumes a significant amount of clinical attention and resources due to the associated psychomotor agitation. Evidence shows those with more severe cases are at a higher risk of death after hospital discharge, are more likely to develop dementia and are more likely to have long-term deficits in cognition. Patients may experience hallucinations and become aggressive posing a risk of physical harm to themselves and the healthcare staff. Common interventions such as mechanical ventilation, sedation and surgery have all been associated with the development of delirium or cognitive dysfunction.

Management of delirium-associated agitation is challenging. Healthcare workers often resort to the use of chemical and physical restraints despite limited evidence and known risks. Delirium care utilizing multi-component strategies is recommended and has been shown to reduce delirium incidence. However, there is a lack of evidence-based, non-pharmacological interventions for delirium-associated agitation. Despite this, guidelines continue to recommend their use. Digital technology-based interventions are becoming more prevalent in the literature. Cognitive stimulation and re-orientation strategies have shown varying levels of success in managing pain, anxiety, or delirium. Where virtual reality (VR) was utilized predominantly as a method for distraction in pediatric and burn populations, clinical trials such as E-CHOISIR have employed VR to expose participants to natural environments in combination with music or hypnosis with outcomes related to anxiety and pain. One of the challenges in applying VR to delirium management is the equipment itself, which is not feasible with actively agitated patients.

The study aimed to determine if using a screen-based digital therapeutic intervention, with a nature-driven imagery delivery that was dynamically responsive to patient agitation, could reduce agitation and reliance on unscheduled medication used in managing delirium-associated agitation. Thus, the use of the MindfulGarden platform, a novel interactive digital therapeutic behavioral monitoring and modification platform aimed at reducing anxiety and agitation associated with hyperactive delirium was studied. The study hypothesized that use of the MindfulGarden behavioral monitoring and modification platform would result in normalization of agitation and delirium scores when used for the management of delirium associated agitation in the adult delirious acute care population compared to standard care alone.

The study type was a clinical trial in which the 70 participants were enrolled. The allocation was randomized with a parallel assignment intervention model used. For the intervention model, participants will be randomized to either exposure to the intervention arm in conjunction with standard care or the control arm and will receive standard care alone.

### Study Design

A single-center, open-label randomized controlled trial was conducted. Participants were admitted to intensive care, high acuity, and cardiac telemetry units. Eligible patients were randomized in a 1:1 ratio to either intervention plus standard of care, or standard of care only.

### Participants:

Participants were adult inpatients with a RASS (Richmond Agitation Sedation Score) +1 or greater for 2 assessments at least 1 hour apart within the 24 hours directly before study enrollment and persisting at the time of enrollment, or equivalent documentation of agitation related to delirium for participants admitted outside of critical care, and ICDSC (Intensive Care Delirium Screening Checklist) 4 at time of enrollment or CAM (Confusion Assessment Method) positive screening. Participants were required to have at least 2 unscheduled medication events in the preceding 24 hours and/or infusion of psychoactive medication (e.g. Dexmedetomidine) for the management of delirium-associated agitation. Participants were excluded if they had a planned procedure or test that precluded participation in the full 4-hour study session, were visually impaired, had significant uncontrolled pain, had RASS less than or equal to 0 at enrollment, refused participation by the responsible physician or were enrolled in another research study which could impact on the outcomes of interest, as evaluated by the Principal Investigator. Participants were recruited with an approved waived consent process.

### Randomization and Blinding:

Eligible patients were randomized using a master randomization list generated by an independent statistician using block permutation (blocks of 2 or 4). Allocation was determined using sequentially numbered opaque envelopes previously filled by a non-research team member and opened after enrolment was confirmed. Blinding to the intervention was not possible due to the nature of the intervention and the logistical constraints of the study.

### Procedures:

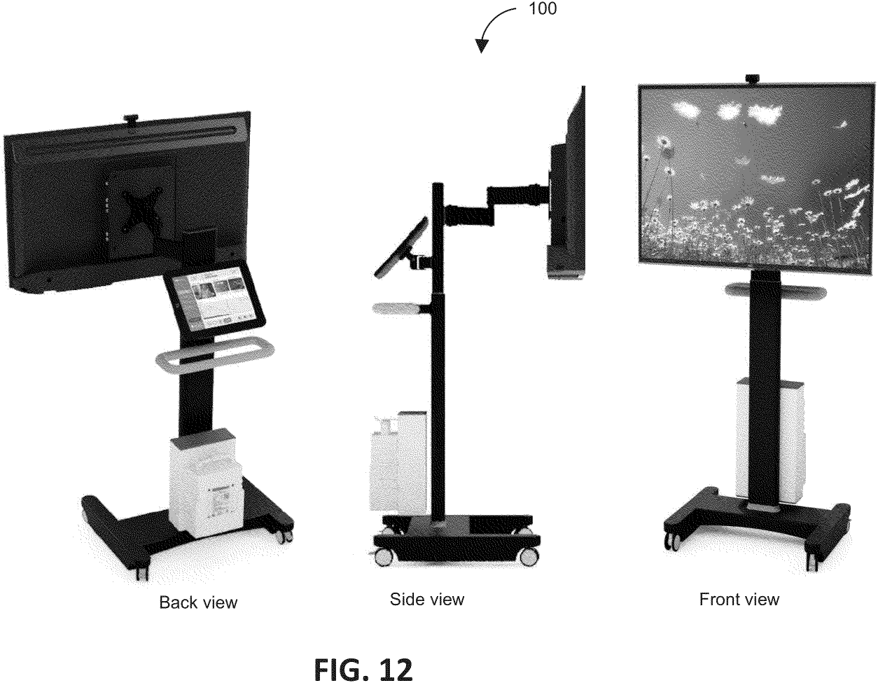

FIG. 12 illustrates the MindfulGarden system 100 according to some embodiments of the present invention. MindfulGarden is a novel, patient-responsive digital behavioral modification platform. The platform utilizes a mobile, high-resolution screen-based digital display with sensor technology. MindfulGarden layers 2D video of real nature imagery with 3D animations in direct response to patient agitation and restlessness for which movement and vocalization are considered the initial surrogate markers. A built-in camera system and microphone use proprietary algorithms to compute the average movement and vocalization input every two seconds and then dynamically adjusts the level of on-screen content when a significant fluctuation in movement and/or vocalization has occurred as compared to the previous two second interval. Animations of growing and receding flowers in addition to butterflies in flight are produced in a volume that is directly responsive to measured patient behavior.

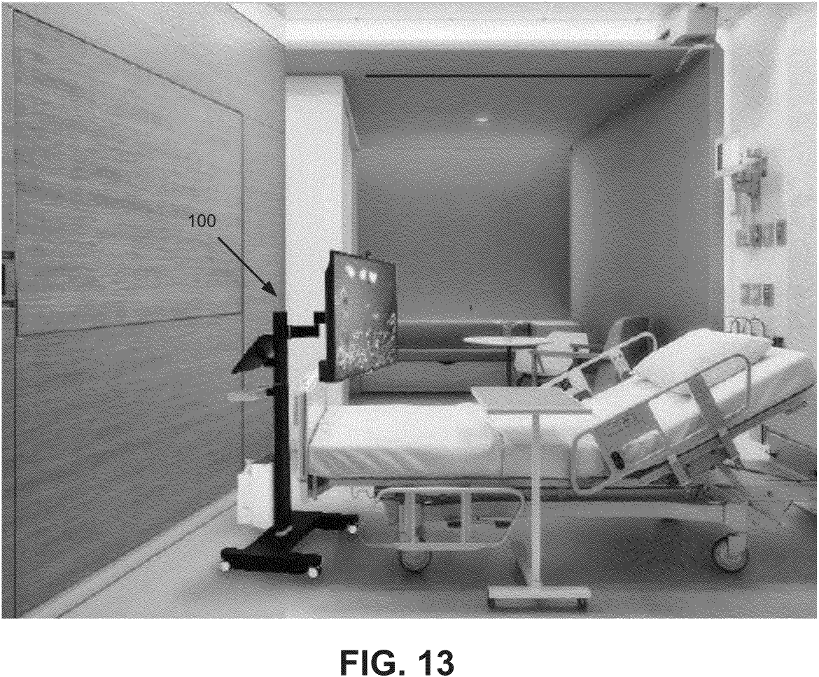

FIG. 13 illustrates an embodiment of the system 100 positioned at the foot of the bed of a patient. As the patient calms and measurable agitation reduces, the volume of animations on screen reduces. Utilization of a digital screen with nature imagery may provide neuro-cognitive and psycho-physiological benefits. Incorporation of an interactive component to the dynamic visual content may be effective as a de-escalation tool for psychomotor agitation.

The platform is mobile, requires no physical attachment to the patient, is implemented with minimal effort and training to healthcare staff, and does not require active management or observation by staff when in use with patients. There is minimal risk of serious complications, and the platform allows the patient the ability to self-direct.

The unit uses an attached camera and microphone to view the patient, and measures sound production in decibels and fluctuations in movement using pixel density. This drives proprietary algorithms to control the on-screen content. The screen is mounted on an articulating arm to allow positional adjustment and a wheeled stand. The MindfulGarden unit utilizes a rechargeable medical-grade battery to allow for further ease of use. In the study, the unit does not physically attach to the patient.

The intervention, MindfulGarden, utilizes a high-definition screen to present a pastoral scene layered with animations of butterflies and flowers blooming. It adjusts the volume of on-screen content in response to movement and sound production, which are surrogate markers of agitation. The screen displays a video of a meadow of flowers that is layered with animations of butterflies in flight and flowers that bloom and recede.

The animations fluctuate in volume driven by the patient agitation measurement algorithms. The animations move at a relaxed speed and are designed to provide a calming experience for the viewer. There is the capability to adjust the responsiveness, speed, and volume of animations; however, all settings were locked at default mid-range settings for this trial. The on-screen experience can adjust the level of brightness according to the time of day to promote natural circadian rhythm. For this trial, all patients received the standard “daylight” settings.

A touchpad attached to the rear of the monitor allowed access to controls and to the standby feature utilized to freeze input for 5-minute intervals without adjusting the current on-screen content. The unit used an automatic restart that could be overridden and started by direct care staff if the provision of care or interaction took less than 5 minutes. The timer could be reactivated without limits. The touchscreen display used a digital readout to allow the user to ensure that the participant was captured within the camera range and measurement zone to limit extraneous activity from activating the intervention.

Sound input was deactivated for those receiving mechanical ventilation to avoid auditory activation from the ventilator and associated alarms. For the purposes of this study, the noise-masking soundtracks were not utilized to be able to determine the effect of the intervention more accurately as a visual therapy.

The display was placed near the foot of the bed for 4 consecutive hours. For the provision of care that required physical interaction with the participant, the device was placed in standby mode for 5-minute intervals. Mechanically ventilated patients had the microphone function disabled to avoid activation by the ventilator and its alarms. The trial was conducted during daytime hours to allow the trial period to be completed within a single nursing shift where possible. Non-pharmacological distraction interventions were halted during the study period, such as other audio-visual interventions (TVs, tablets, or music) in both arms. Reorientation by staff, use of whiteboards, clocks, family presence, repositioning, mobilization, physiotherapy, and general nursing care continued uninterrupted throughout the study period.

Anonymized patient and session data is encrypted and logged to a secure database on the unit, providing dashboard analytics. All Wi-Fi and Bluetooth connectivity were disabled and recording functions were turned off for the purposes of this trial to ensure patient privacy and anonymity.

### Outcomes:

As discussed in more detail below, primary outcomes were measured by Agitation scores in a time frame of four hours, primarily the Richmond Agitation Sedation Score. Richmond Agitation Sedation Score is a validated and standardized scoring system ranging from -5 (deeply sedated) to 0 (awake and calm) to +4 (combative). Scores are measured hourly from study start, one hour post intervention and at the start of the following nursing shift.

Secondary outcome measures were also employed. Secondary outcome measures included:
1. Use of unscheduled medications for the management of delirium associated agitation [ Time Frame: 4 hours ]. Incidence of unscheduled or “PRN” medication use for the management of delirium associated agitation throughout the 4-hour study period.
2. Delirium Scores [ Time Frame: 4 hours ]. Delirium scores were measured using the Intensive Care Delirium Screening Checklist. Delirium score is a range of zero to 8 with scores above or equal to 4 being diagnostic for the presence of delirium and higher scores being indicative of added severity of symptoms. Intensive Care Delirium Screening Checklist will be measured at study initiation, after 2 hours, at study completion (4 hours) and the start of the following nursing shift.
3. Richmond Agitation Sedation Scale of zero [ Time Frame: 4 hours ]. The proportion of patients achieving a Richmond Agitation Sedation Scale score of zero throughout the study period. Score range is -5 to +4 with a score of zero indicating the patient is awake and calm. Negative scores indicate deeper sedation, positive scores reflect agitation.
4. Physical Restraint Use [Time Frame: 4 hours]: The proportion of participants with physical restraints in use throughout the study period and the length of time of restraints in use.
5. Incidence of Unplanned Line removal [ Time Frame: 4 hours ]: The incidence of unplanned removal of lines or tubes by the study participant (endotracheal tubes, nasogastric tubes, oral-gastric tubes, central venous lines, peripheral intravenous lines, urinary catheters, arterial lines) throughout the study period.
6. PRN medication use in the 2 hours post study [ Time Frame: 2 hours ]: The incidence of unscheduled medication administration for the management of delirium behaviors in the 2 hours following the study or intervention period.
7. Movement Count Average [ Time Frame: 4 Hours ]: Those in the intervention arm had generated activity logs stored within the device units. The movement count average is calculated by comparing the difference in pixel density from the previous frame to the current one. The resulting value is then averaged over the collected frames and returned as a decimal percentage of change. Values are between 0 and 1 with 0 showing the lowest amount of activity and one the highest.
8. Physiological data [ Time Frame: 4 hours ]: Basic physiological data were collected and analyzed from nursing records and for a smaller proportion directly from telemetry monitors where available to compare between arms as well as to evaluate trends over the course of the study period. Parameters include heart rate, mean arterial blood pressure, respiratory rate, oxygen saturation and use of vasopressors
9. Heart rate variability [ Time Frame: 6 hours ]: For a small subset of the overall population ECG data was collected to assess differences in heart rate variability between study arms measured as pNN50 and RMMSD. Five minute ECG recordings will be taken hourly starting one hour before the study period until one hour post timed to match agitation and delirium scores.

Other outcome measures included a survey of caregivers and a survey of family members.

The primary outcome was mean agitation (RASS) scores over the study period with RASS measured preexposure and every hour thereafter until one hour post the 4-hour intervention period. Secondary outcomes included the proportion of participants receiving unscheduled pharmacological interventions for the management of delirium-associated agitation during the 4-hour study period, delirium scores (ICDSC at study initiation, 2 hrs, and 4 hrs), the proportion of patients achieving target RASS of 0 or -1 (indicating awake and calm to mildly drowsy), use of physical restraints, the incidence of unplanned removal of lines, tubes or equipment by participants throughout the study period and time to event from the start of the study period of these events, and the proportion of participants receiving unscheduled pharmacological intervention in the 2-hours post-intervention.

### Data Collection

For the outcomes of RASS and ICDSC scores, bedside nurses conducted assessments and documented scores on paper-based forms which were then collected by research staff. Nursing staff in critical care and high acuity areas used these scoring systems routinely in patient assessments. For participants enrolled in cardiac telemetry wards, observations were conducted by trained research personnel in collaboration with ward nurses.

### Sample Size:

Based on clinical experience in the ICU, it was anticipated that over a period of 4 hours approximately 70% of agitated delirious patients would receive unscheduled medications for delirium. It was anticipated the intervention would decrease this by a 50% relative reduction from 70% incidence to 35%. The required sample size was calculated to be 31 patients per arm, with a power of 80% and a significance level of 0.05. This was increased slightly in recognition that it was an estimated effect size and is supported by previous literature.

### Statistical Plan:

Descriptive statistics are presented using Mean (+/-SD) or Mean (IQR) with proportions being represented as the total number and percentage n(%). Within-group changes were tested using parametric paired t-test or non-parametric Wilcoxon-signed rank and between groups using unpaired-ttest or Mann Whitney-U. Proportions were tested using Chi-sq or Fisher’s Exact. The primary outcome of RASS scores was further analyzed in a multivariate linear regression model with the treatment arm as the primary explanatory variable and adjusting for age, sex, pre-exposure RASS score and a surgical or medical cause of admission as was ICDSC. Yes/No unscheduled drug administration was analyzed with multivariate logistic regression. An unscheduled drug event included the unscheduled use of antipsychotics, sedatives, narcotics and where participants were on continuous infusions of medications (e.g.: dexmedetomidine) a ³20% increase in dose was considered an unscheduled event. A-priori subgroup analyses of mean RASS scores were planned to ascertain what may be the optimal target population for the intervention and including the presence of traumatic brain injury (TBI), mechanical ventilation at the time of the trial, delirium >24 hrs, and medical or surgical cause of admission (Kruskal-Wallis, see Table 2.0). A p-value <0.05 was considered significant for all results. The main statistical analysis for the outcomes of RASS, regression and subgroup analyses was conducted by an independent statistician using SAS Version 9.1. Secondary outcomes were analyzed using GraphPad Prism Version 9.4.1. This study is registered with ClinicalTrials.gov, NCT04652622.

### Results:

A total of 73 participants were recruited between March 16th, 2021 and January 5th, 2022 with 70 included in the final analysis (See FIG. 1.0). Three participants were excluded after randomization, two before study start due to changes to the course of clinical care and one duplicate enrolment. Participants were recruited from critical care (n=65) and high acuity cardiac telemetry wards (n=5). See Table One for further details on patient demographics and characteristics.

TABLE One

Demographics and characteristics of included participants:

| Demographic and medical characteristics: | Control (n=35) | Intervention (n=35) |
|---|---|---|
| Age: mean (range) | 61.5 (20-89) | 60.3 (19-86)) |
| Sex: Male n(%) | 21 (60.0) | 27 (77.1) |
| BMI: mean (SD) | 26.6(4.83) | 27.5(6.02) |
| Renal replacement therapy n(%) | 3(8.6) | 9(25.7) |
| COPD n(%) | 7(20) | 4(11.4) |
| Underlying brain health condition (TBI, stroke, dementia) n(%) | 11(31.4) | 14(40) |
| Psychiatric history n(%) (eg depression, anxiety disorder, bi-polar) | 11(31.4) | 12(34.3) |
| Days since first delirium diagnosis: mean (SD) | 3.9(3.3) | 4.5(3.5) |
| Substance use history n(%) | 8(22.9) | 9(25.7) |
| APACHE IV Score mean(SD) | 37.1(16.66) | 43.7(16.7) |
| Covid19 Positive n(%) | 10(28.6) | 8(22.9) |
| Diabetic n(%) | 6(17.1) | 14(40.0) |
| Mechanical ventilation at the time of study | 7 (20.0%) | 5 (14.3%) |
| Admission Diagnosis: | | |
| Trauma n(total %) | 6(17.1) | 4{11.4) |
| Traumatic brain injury n(%) | 5(14.3) | 3(8.6) |
| Neurological (non-traumatic) n(%) | 4(11.4) | 6(14.3) |
| Sepsis n(%) | 2(5.7) | 3(8.6) |
| Cardiovascular n(%) | 11(31.4) | 11(31.4) |
| Respiratory n(%) | 10(28.6) | 9(25.7) |
| Other n(%) | 2(5.7) | 2(5.7) |

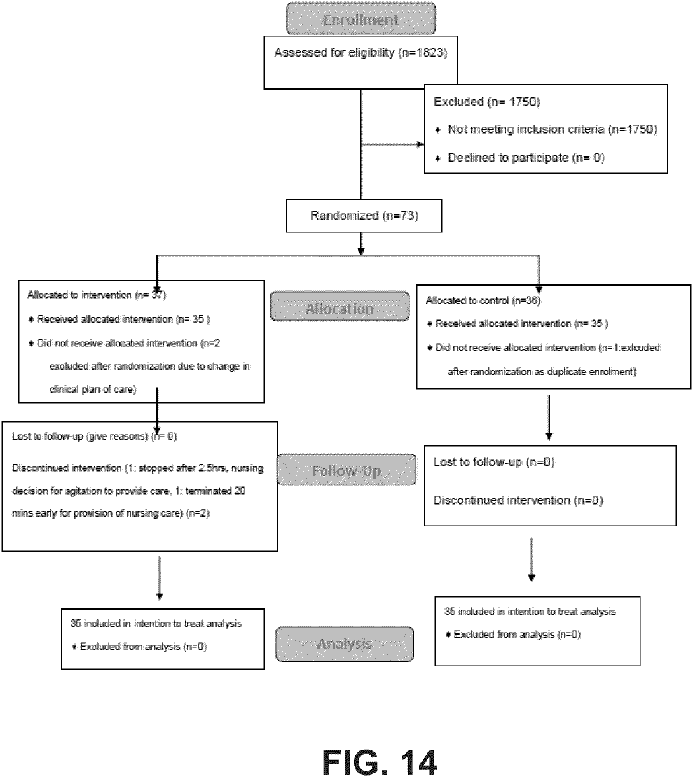

In the intervention arm, 2 participants did not complete the full 4-hour exposure, one ended 20 minutes early and one after 2.5 hours of exposure due nursing decisions for the necessary provision of care. All participants were analyzed according to intention to treat principles as illustrated in FIG. 14.

For logistical reasons one participant in the intervention arm did not have full data acquisition. Missed data points include RASS and ICDSC scores at hours 1,3, and 4. Mean RASS scores in the intervention and control arms were not significantly different at study initiation (1·6 (0·95) vs 1·2 (0·95) respectively, p=0·27).

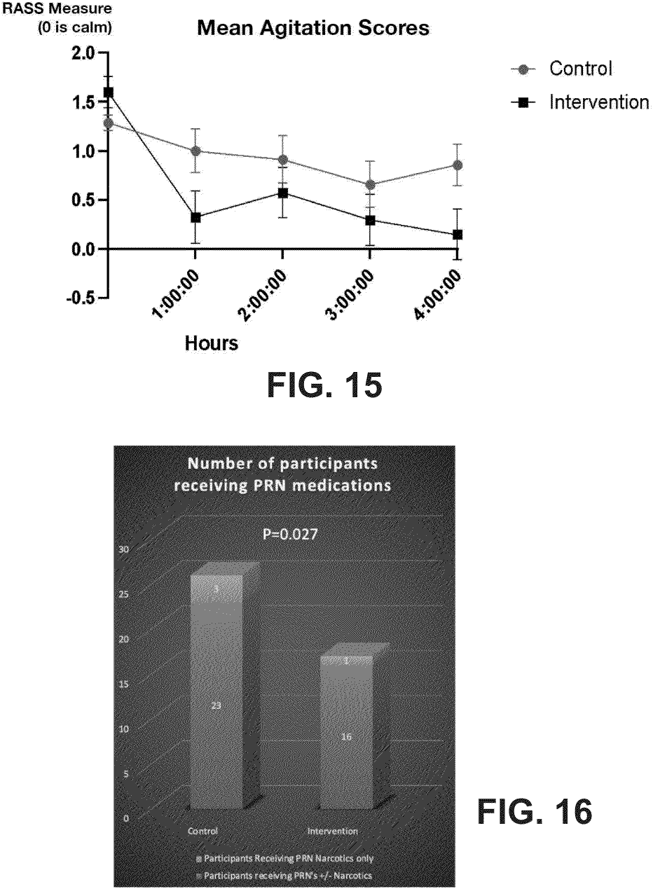

FIG. 15 illustrates the Mean Agitation Scores of participants experiencing intervention as opposed to the control group. Participants in the intervention group wherein the MindfulGarden behavior monitoring and modification platform of the present invention was used experienced a significant reduction in Mean Agitation Scores as compared to the control group. The error bars show the standard error of the mean (SEM) Hour 0 denotes pre-exposure scores. The dotted line at hour 4 shows the interventional period end.

FIG. 16 illustrates the number of participants receiving PRN medications displayed as all patients represented as a % in each study arm that received unscheduled medication by hour with “post” including in the two-hours post-study completion. The intervention group (MindfulGarden) showed an absolute decrease of 25.7% in administration of any PRN medication. Overall, the platform showed a 30% reduction in Behavioral and Psychological Symptoms of Dementia (BPSDs) in patients in long-term care. The results of the 70-patient RCT (NCT04652622) addressing use of the MindfulGarden platform in reducing levels of measurable agitation in acute care delirium patients (compared to standard care) indicates significant positive results.

At hour one post-study initiation mean RASS scores were significantly decreased from baseline scores in the intervention group but not the control group 0·3(1·55) p<0·0001 vs ·0(1·33)p=0·15 respectively. This corresponded with a significant difference in the proportion of participants showing a reduction of RASS at hour-1 with 24(70.6%) intervention vs 14(40%) control (RR 0·57, 95%CI 0·35-0·88, p=0·01). This effect was maintained for the main outcome of mean RASS across the 4-hour study period which was significantly lower in the intervention arm (0·3 (0·85) vs 0·9 (0·93), p=0·01). Multivariate linear regression showed only study arm allocation was associated with mean 4-hour RASS over the duration of the study (RR -0.48, 95%CI -0.92 - -0.03,p=0·04).

The proportion of total RASS measurements during the 4-hour intervention period at 0 (calm and awake) or -1 (mildly drowsy) was significantly higher in the intervention group 63(46%) than in the control group 41 (29%) respectively (RR 0·64: 95%CI 0·46-0·87, p=0·004). 15 participants in each group achieved a RASS of zero at some point during the 4-hour study period. The proportion of RASS measures at 0 at any time over the 4-hour study period was higher in the intervention arm but this was not statistically significant, 32(23 ·4%) vs 26(18·6%) (RR 0·8, 95%CI: 0·50-1·25, p=0·33).

A-priori planned subgroup analyses of the effect of the intervention on specific groups were conducted. Participants showed lower mean RASS scores in the intervention arm who were not mechanically ventilated at the time of the study (p= 0·003), had a diagnosis of delirium >24 hrs (p=0·02), did not have a traumatic brain injury (TBI) (p=0·02) and had a medical cause of admission (p<0·0001). See Table 2.0 for a full breakdown of these results.

TABLE 2.0

Subgroup Analysis of RASS Hours 1-4

| | Control | Intervention | p-value |
|---|---|---|---|
| TBI-No | n=30 | n=32 | 0·02¹ |
| Mean | 0·8(0·95) | 0·4(0·87) | |
| Med | 0·9(0·5-1·5) | 0·4(-01-0·8) | |
| TBI-Yes | n=5 | n=3 | 0·17¹ |
| Mean | 1·1(0·91) | 0·1(0·63) | |
| Med | 0·8(0·5-1·5) | 0·0(-0·5-0·8) | |
| MV-no | n=28 | n=30 | 0·003¹ |
| Mean | 0-9(0·83) | 0·3(0·76) | |
| Med | 0·8(0·5-1·5) | 0·4(0·0-0·8) | |
| MV-Yes | n=7 | n=5 | 0·68¹ |
| Mean | 0·6(1·31) | 0·7(1·36) | |
| Med | 1·0(-0·5-1·8) | 0·4(0·0-0·8) | |
| Delirium>24hrs-Yes | n=15 | n=11 | 0·02¹ |
| Mean | 1·0(0·78) | 0·1(0·74) | |
| Med | 0·9(0·5-1·8) | 0·4(0·89) | |
| Medical Admit | | n=27 | <0.0001¹ |
| Mean | 1·1(0·80) | 0·1(0·62) | |
| Med | 1·1(0·5-1·8) | 0·3(-0·3-0·5) | |
| Surgical Admit | n=13 | n=8 | 0·26¹ |
| Mean | 0·4(1·02) | 1·0(1·17) | |
| Med | 0·5(0·0-0·8) | 1·0(0·1-2·1) | |

1: Kruskal-Wallis p-value.

For the secondary outcome of unscheduled medication use, a significant difference was shown in the proportion of participants receiving unscheduled medication throughout the four hours of the study period with 17(48·6%) intervention vs 26(74·3%) control respectively, an absolute difference of 25·7% (RR 1·53, 95% CI 1·049-2·332, NNT 4, p=0·03). Mean drug events per participant were not significantly different between the intervention and control groups, 1.26 (1·84) vs 1·69 (1·62) respectively, p=0·30. Multivariate analysis did not show any association of age or gender on unscheduled medication use although inclusion in the intervention arm shows a trend toward significance at p=0·06. In the 2-hour post-trial period the proportion receiving unscheduled medications was not significant between study arms, intervention 16(45·7%) vs control 17(48·6%),(RR 1·06 95% CI:0·64-1·76 p=0·8). Median delirium scores using ICDSC were similar pre-exposure in the intervention and the control groups, 5·0 (4·0-6·0) vs 5·0 (4·0-6·0) respectively, p=0·62. Similarly, they were not significantly different between the intervention and control groups at hour 2, Med(IQR) 4·0(4·0-5·0) vs 5·0 (4·0-5·0), p=0·65 and hour-4, 5·0 (4·0-6·0) vs 4·0 (4·0-5·0) respectively, p=0·46. In multivariate linear regression, age was positively associated with ICDSC score at hour 2 (RR 0·02 95%CI: 0·0-0·04, p=0·02), but not at hour-4, (RR 0.0195%CI: 0·01-0·03, p=0·15) and there was no association Of ICDSC scores and gender or study arm. (See Table 3.0).

TABLE 3.0

Regression Analysis

| | | | | |
|---|---|---|---|---|
| | Age | Female gender | RASS Pre-exp | Intervention vs Control |
| Mean RASS¹ | -0·01(-0·01-0·01), p=0·40 | 0·11(-0·36-0·58), p=0·65 | -0·13(-0·43-0·17), p=0·39 | -0·48(-0·92-0·03), p=0·04 |
| Use of Unscheduled Medication (Y/N)² | 1(0-97-1-03), p=0·91 | 2·47(0·76-8·05), p=0·13 | | 0-36(0-13-1-02), p=0·06 |
| ICDSC Hour 2¹ | 0·02 (0·0, 0-04), p=0·02 | 0·3 (-0·45, 1-06), p=0·43 | | -0·1 (-0·81, 0·6), p=0·77 |
| ICDSC Hour 4¹ | 0·01 (-0·01, 0·03), P=0·15 | -0-07 (-0·8, 0·66), P=0·85 | | 0·29 (-0-39, 0-97), p=0·39 |

1: Multivariate linear regression, results RR (95% CI), 2: Multivariate logistic regression, results OR (95%CI)

Use of physical restraints was common at study start, 26(74·3%) intervention vs 29(82·9%) control (RR 1.1, 95%CI: 0·86-1·47,p=0·38) and at one-hour post-trial completion, 24(69%)intervention vs 30(86%) control (RR1·25, 95%CI: 0·97-1·68, p=0·09). Restraint use tended to be continuous when used. The proportion of participants reported to have an unplanned line or equipment removal (such as patient pulling out IV’s or nasogastric tubes) was not significant between arms, 1(2·9%)intervention vs 4(11.4%) in the control (RR 4·0, 95%CI 0·63-26·0, p=0·36) with 1 vs 5 total events respectively. Mean(SD) time in minutes to the event from the start of the intervention period was intervention 150 (single event) vs. control 104·2(86) which was not significant. (See online supplement) No specific harms from the intervention were observed during the study period.

### Discussion:

The study results show a significant reduction in agitation with exposure to the digital calming intervention that was maintained over the 4-hour study period. This reduction in RASS was achieved with fewer potentially toxic unscheduled medications. These findings are important as they set the foundation for digital therapeutics in delirious, hospitalized patients. What may be just as important as the decrease in mean RASS scores, is that 70% of participants exposed to the intervention had a reduction in RASS at hour one. A reduction in RASS is perhaps more significant than achieving a goal RASS of zero or -1.

A reduction of more than 25% in unscheduled medication use may have clinical benefits and is an important finding. The simultaneous reduction in RASS and unscheduled medication use for managing agitation gives more validity to the inference that patients were being calmed and distracted by the intervention. These reductions could have significant downstream benefits to patients by avoiding complications and reducing the burden on nursing staff. Although not studied, it may also reduce distressing aspects of the patient’s experience and may influence the course of delirium as physical and chemical restraints may in themselves contribute to delirium. While physical restraint use was high overall, this may be more reflective of having conducted the trial during the Covid19 pandemic with significant strain on nursing resources.

The a-priori planned subgroup analysis provides some insight as to which groups may benefit most from this intervention, although this must be interpreted with caution due to the small numbers in some subgroups. It seems reasonable that patients who were not intubated may derive the most benefit as the device could utilize vocalization as well as movement as markers of agitation. Interestingly, the intervention was more effective in patients without TBI, although there was a trend towards an effect in those with head injuries and this may be a function of the small sample size. It is not clear why patients with a medical reason for admission were more responsive to the calming effects of the intervention. Although this too suffered from a small surgical sample size. A final subgroup that showed significantly more response to the intervention is those with a diagnosis of delirium of greater than 24 hours. This group potentially had a more established pattern of agitation that was somewhat resistant to traditional non-pharmacological interventions. Likely transient delirium may not require the same degree of intensity of interventions that more established delirium does. While agitation scores were reduced, measures of delirium were not, with no significant change in ICDSC scores over the study period. This may show that while the intervention is effective in reducing agitation, it was not effective at reversing or reducing measurable delirium. This seems reasonable as the intervention likely distracts and calms the patient but does not change the underlying cause of delirium.

This trial’s most notable limitation is it being open-labelled and reliant on direct care nursing staff to score and report outcomes such as agitation scores. While this is part of the normal conduct of care, the inability to blind providers or outcome assessors to the intervention introduced possible bias. Similarly, a degree of Hawthorne effect may be present by staff self-modulating their response to patient agitation in the use of unscheduled medications knowing their practice was being observed. Indeed, the initial primary outcome was planned to be unscheduled drug use by the bedside nurses. However, this was felt too sensitive to potential bias and was changed to RASS scores within 4 months of study initiation and at 19% recruitment completed. This was before any data was accessed or analyzed. Although this change in the primary outcome should be considered a weakness, it was determined to be reasonable as both were a-priori planned outcome measures, they were already being gathered and the change was to what we felt provided a more rigorous primary outcome.

Interestingly, both outcomes of RASS and unscheduled medication use showed a significant improvement with the intervention thus mitigating this potential weakness. The overall sample size is likely underpowered for some subgroup analyses. The interactive component of the intervention cannot be definitively shown to have a causative effect on the outcomes of interest. A comparison of a TV or intervention without the interactive component may be required to understand the effect more clearly, as well as the mechanism of action. While this study was completed in a predominantly critical care environment, it is reasonable to expect that the intervention would be effective, or even potentially amplified, in the general hospital population with a lower nurse-to-patient ratio.

There is a clear need for effective non-pharmacological interventions for the management of delirium. The study provides the initial work demonstrating that interactive digital therapeutics are an effective non-pharmacological approach to manage agitated delirium. It may provide a strategy to reduce the burden of nursing care and improve resource utilization. Initial validation data from use of MG in a long-term care dementia population showed a 46% positive rating from staff in successful agitation reduction when MG was used as a behavioral “crash cart” for management of self manifesting agitated behaviors with a 0% negative rating (13 exposure events for 7 participants).

Although this study was not powered to show clinical outcomes, there are potential benefits in terms of length of stay, morbidity, and economic burden to healthcare systems. Interactive digital therapeutics for delirium provide a novel adjunct to agitation management while potentially reducing the risk profile associated with traditional strategies.

This novel nonpharmacological intervention may improve patient outcomes and reduce nursing burden although the optimal application of this new tool remains to be determined through future research. These results indicate the platform of the instant invention may be used to reduce delirium agitation scores and sedation levels.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for providing automated behavior monitoring and modification in a patient, the system comprising:
an audio/visual device configured to present audible and/or visual content to a patient exhibiting one or more disruptive behaviors associated with a mental state;
one or more sensors configured to continuously capture patient activity data during presentation of the audible and/or visual content, the patient activity data comprising at least one of patient motion, vocalization, and physiological readings; and
a computing system operably associated with the audio/visual device and configured to control output of the audible and/or visual content therefrom based, at least in part, on the patient activity data, wherein the computing system is configured to:
receive and analyze, in real time, patient activity data from the one or more sensors and determine a level of increase or decrease in patient activity over a period of time; and
dynamically adjust a level of output of the audible and/or visual content from the audio/visual device to correspond to the determined level of increase or decrease in patient activity.

2. The system of claim 1, wherein an increase in patient activity comprises at least one of increased patient motion, increased vocalization, and increased levels of physiological readings.

3. The system of claim 2, wherein the computing system is configured to increase a level of output of audible and/or visual content to correspond to an increase in patient activity.

4. The system of claim 3, wherein an increased level of output of audible and/or visual content comprises at least one of: an increase in an amount of visual content presented to the patient; an increase in a type of visual content presented to the patient; and increase in movement of visual content presented to the patient; an increase in a decibel level of audible content presented to the patient; an increase in frequency and/or tone of audible content presented to the patient; and an increase in tempo of audible content presented to the patient.

5. The system of claim 1, wherein a decrease in patient activity comprises at least one of decreased patient motion, decreased patient vocalization, and decreased levels of patient physiological readings.

6. The system of claim 5, wherein the computing system is configured to decrease a level of output of audible and/or visual content to correspond to a decrease in patient activity.

7. The system of claim 6, wherein a decreased level of output of audible and/or visual content comprises at least one of: a decrease in an amount of visual content presented to the patient; a decrease in a type of visual content presented to the patient; a decrease in movement of visual content presented to the patient; a decrease in a decibel level of audible content presented to the patient; a decrease in frequency and/or tone of audible content presented to the patient; and a decrease in tempo of audible content presented to the patient.

8. The system of claim 1, wherein the computing system is configured to dynamically adjust levels of output of the audible and/or visual content based on adjustable predefined ratios applied to patient activity data.

9. The system of claim 1, wherein the patient motion comprises facial expressions, physical movement, and/or physical gestures.

10. The system of claim 1, wherein the physiological readings comprise at least one of the patient’s: body temperature, heart rate, heart rate variability, blood pressure, respiratory rate, respirator depth, skin conductance, and oxygen saturation.

11. The system of claim 1, wherein the one or more disruptive behaviors comprise varying levels of agitation, distress, and/or confusion associated with the mental state.

12. The system of claim 11, wherein the disruptive behaviors are associated with delirium.

13. The system of claim 12, wherein each of the varying levels of agitation, distress, and/or confusion is associated with a measured Richmond Agitation Sedation Score and/or a Delirium Score.

14. The system of claim 1, wherein the one or more sensors comprises one or more cameras, one or more motion sensors, one or more microphones, and/or one or more biometric sensors.

15. The system of claim 1, wherein the audible and/or visual content presented to the patient comprises sounds and/or images.

16. The system of claim 15, wherein the images comprise two-dimensional (2D) video layered with three-dimensional (3D) animations.

17. The system of claim 15, wherein the images comprise nature-based imagery.

18. The system of claim 15, wherein content in the images is synchronized to the time of day in which the images are presented to the patient.

19. The system of claim 15, wherein the sounds are noise-cancelling and/or noise-masking.
